SEQUENCE LISTING (77 sequences)

SEQ ID NO: 1 - protein, 6 aa, Artificial Sequence
  zinc finger
QSRYTT

SEQ ID NO: 2 - protein, 6 aa, Artificial Sequence
  zinc finger
QSRYTT

SEQ ID NO: 3 - protein, 7 aa, Artificial Sequence
  zinc finger
TGEKPFA

SEQ ID NO: 4 - protein, 6 aa, Artificial Sequence
  zinc finger
RSDNRA

SEQ ID NO: 5 - protein, 6 aa, Artificial Sequence
  zinc finger
RSDETR

SEQ ID NO: 6 - protein, 6 aa, Artificial Sequence
  zinc finger
FQSGIQ

SEQ ID NO: 7 - protein, 6 aa, Artificial Sequence
  zinc finger
HKRNTD

SEQ ID NO: 8 - protein, 6 aa, Artificial Sequence
  zinc finger
DQSALG

SEQ ID NO: 9 - protein, 6 aa, Artificial Sequence
  zinc finger
TKQNTH

SEQ ID NO: 10 - protein, 6 aa, Artificial Sequence
  zinc finger
QLATSY

SEQ ID NO: 11 - protein, 6 aa, Artificial Sequence
  zinc finger
RNGNTR

SEQ ID NO: 12 - protein, 6 aa, Artificial Sequence
  zinc finger
YQPNIN

SEQ ID NO: 13 - protein, 6 aa, Artificial Sequence
  zinc finger
RSRSAK

SEQ ID NO: 14 - protein, 6 aa, Artificial Sequence
  zinc finger
HGPSKK

SEQ ID NO: 15 - protein, 6 aa, Artificial Sequence
  zinc finger
WASQSR

SEQ ID NO: 16 - protein, 6 aa, Artificial Sequence
  zinc finger
WMNVKR

SEQ ID NO: 17 - protein, 6 aa, Artificial Sequence
  zinc finger
WQAVAR

SEQ ID NO: 18 - protein, 6 aa, Artificial Sequence
  zinc finger
WSQCRR

SEQ ID NO: 19 - protein, 6 aa, Artificial Sequence
  zinc finger
WSSIIR

SEQ ID NO: 20 - protein, 6 aa, Artificial Sequence
  zinc finger
WVSTTR

SEQ ID NO: 21 - protein, 6 aa, Artificial Sequence
  zinc finger
WNHGRR

SEQ ID NO: 22 - protein, 6 aa, Artificial Sequence
  zinc finger
CPNARH

SEQ ID NO: 23 - protein, 6 aa, Artificial Sequence
  zinc finger
WNSALR

SEQ ID NO: 24 - protein, 6 aa, Artificial Sequence
  zinc finger
WANQHR

SEQ ID NO: 25 - protein, 6 aa, Artificial Sequence
  zinc finger
QSPAKY

SEQ ID NO: 26 - protein, 6 aa, Artificial Sequence
  zinc finger
WPVRAR

SEQ ID NO: 27 - protein, 6 aa, Artificial Sequence
  zinc finger
YMPHRR

SEQ ID NO: 28 - protein, 6 aa, Artificial Sequence
  zinc finger
WCVSKR

SEQ ID NO: 29 - protein, 6 aa, Artificial Sequence
  zinc finger
WFSGSR

SEQ ID NO: 30 - protein, 6 aa, Artificial Sequence
  zinc finger
WSNLKR

SEQ ID NO: 31 - protein, 6 aa, Artificial Sequence
  zinc finger
WCVTKR

SEQ ID NO: 32 - protein, 6 aa, Artificial Sequence
  zinc finger
WPLRNR

SEQ ID NO: 33 - protein, 6 aa, Artificial Sequence
  zinc finger
WDSSNR

SEQ ID NO: 34 - protein, 6 aa, Artificial Sequence
  zinc finger
WSASDR

SEQ ID NO: 35 - protein, 6 aa, Artificial Sequence
  zinc finger
HGPSHK

SEQ ID NO: 36 - protein, 5 aa, Artificial Sequence
  zinc finger
WSSIR

SEQ ID NO: 37 - protein, 6 aa, Artificial Sequence
  zinc finger
WVNSWR

SEQ ID NO: 38 - protein, 6 aa, Artificial Sequence
  zinc finger
WVQNRR

SEQ ID NO: 39 - protein, 6 aa, Artificial Sequence
  zinc finger
LPPTKH

SEQ ID NO: 40 - protein, 6 aa, Artificial Sequence
  zinc finger
HSPPKK

SEQ ID NO: 41 - protein, 6 aa, Artificial Sequence
  zinc finger
LSGGRK

SEQ ID NO: 42 - protein, 6 aa, Artificial Sequence
  zinc finger
WNSASR

SEQ ID NO: 43 - protein, 6 aa, Artificial Sequence
  zinc finger
WCNVKR

SEQ ID NO: 44 - protein, 6 aa, Artificial Sequence
  zinc finger
WFQARR

SEQ ID NO: 45 - protein, 6 aa, Artificial Sequence
  zinc finger
WSNLHR

SEQ ID NO: 46 - protein, 6 aa, Artificial Sequence
  zinc finger
WVTTMR

SEQ ID NO: 47 - protein, 6 aa, Artificial Sequence
  zinc finger
WKAGDR

SEQ ID NO: 48 - protein, 6 aa, Artificial Sequence
  zinc finger
NAPSRK

SEQ ID NO: 49 - protein, 6 aa, Artificial Sequence
  zinc finger
WHAGTR

SEQ ID NO: 50 - protein, 6 aa, Artificial Sequence
  zinc finger
YKPNRR

SEQ ID NO: 51 - protein, 6 aa, Artificial Sequence
  zinc finger
ARNDSR

SEQ ID NO: 52 - protein, 6 aa, Artificial Sequence
  zinc finger
NSTALQ

SEQ ID NO: 53 - protein, 6 aa, Artificial Sequence
  zinc finger
RTNSQD

SEQ ID NO: 54 - protein, 6 aa, Artificial Sequence
  zinc finger
QIGSQF

SEQ ID NO: 55 - protein, 6 aa, Artificial Sequence
  zinc finger
NINPQY

SEQ ID NO: 56 - protein, 5 aa, Artificial Sequence
  zinc finger
ARNDS

SEQ ID NO: 57 - protein, 6 aa, Artificial Sequence
  zinc finger
DTNRKH

SEQ ID NO: 58 - protein, 6 aa, Artificial Sequence
  zinc finger
GLASQD

SEQ ID NO: 59 - protein, 6 aa, Artificial Sequence
  zinc finger
HTNQKT

SEQ ID NO: 60 - protein, 6 aa, Artificial Sequence
  zinc finger
YSTALQ

SEQ ID NO: 61 - protein, 6 aa, Artificial Sequence
  zinc finger
RTNSQD

SEQ ID NO: 62 - protein, 6 aa, Artificial Sequence
  zinc finger
RTNGNR

SEQ ID NO: 63 - protein, 6 aa, Artificial Sequence
  zinc finger
RTEDSR

SEQ ID NO: 64 - protein, 4 aa, Artificial Sequence
  zinc finger
TDSR

SEQ ID NO: 65 - protein, 21 aa, Artificial Sequence
  zinc finger
ACDEVFGHIKLMNPQRSTVWY

SEQ ID NO: 66 - protein, 7 aa, Artificial Sequence
  zinc finger
RSDNLRA

SEQ ID NO: 67 - protein, 7 aa, Artificial Sequence
  zinc finger
QLATLSN

SEQ ID NO: 68 - protein, 7 aa, Artificial Sequence
  zinc finger
DQSNLTR

SEQ ID NO: 69 - protein, 7 aa, Artificial Sequence
  zinc finger
FQSGLIQ

SEQ ID NO: 70 - protein, 7 aa, Artificial Sequence
  zinc finger
HKRNLTD

SEQ ID NO: 71 - protein, 7 aa, Artificial Sequence
  zinc finger
DQSALLG

SEQ ID NO: 72 - protein, 7 aa, Artificial Sequence
  zinc finger
TKQNLTH

SEQ ID NO: 73 - protein, 7 aa, Artificial Sequence
  zinc finger
QLATLSY

SEQ ID NO: 74 - protein, 7 aa, Artificial Sequence
  zinc finger
RNGNLTR

SEQ ID NO: 75 - protein, 7 aa, Artificial Sequence
  zinc finger
YQPNLIN

SEQ ID NO: 76 - protein, 6 aa, Artificial Sequence
  zinc finger
QLASTN

SEQ ID NO: 77 - protein, 6 aa, Artificial Sequence
  zinc finger
DQSNTR

(19) United States
(12) Patent Application Publication
(10) Pub. No.: US 2023/0167436 A1
(43) Pub. Date: Jun. 1, 2023

(54) COMPOSITIONS AND METHODS FOR IDENTIFICATION OF ZINC FINGERS

(71) Applicant: New York University, New York, NY (US)

(72) Inventor: Marcus NOYES, Monmouth Junction, NJ (US)

(21) Appl. No.: 17/997,467

(22) PCT Filed: Apr. 30, 2021
(86) PCT No.: PCT/US2021/030267

§ 371 Date: Oct. 28, 2022

Related U.S. Application Data
(60) Provisional application No. 63/018,289, filed on Apr. 30, 2020.

(51) Int. Cl. C12N 15/10

(52) U.S. Cl. CPC C12N 15/1086; C07K 2319/81

(57) ABSTRACT

Provided are improved compositions and methods that are used for identifying interacting zinc fingers in a zinc finger and DNA sequence context. The compositions and methods provide a comprehensive approach that takes into account the effect of adjacent zinc fingers, in part by expanding the repertoire of F2 fingers that are varied at amino acid position 6.

### CROSS REFERENCE TO RELATED APPLICATIONS

This application claims priority to U.S. provisional patent application No. 63/018,289, filed Apr. 30, 2020, the entire disclosure of which is incorporated herein by reference.

### STATEMENT REGARDING FEDERALLY SPONSORED RESEARCH

This invention was made with government support under grant no. R01GM118851 and R01GM133936 awarded by the National Institutes of Health. The government has certain rights in the invention.

### FIELD

The present disclosure relates to identification of amino acid sequences of zinc fingers that bind to DNA targets in a nucleotide and adjacent zinc finger context specific manner.

### BACKGROUND

The Cys₂His₂ zinc finger (ZF) is the most common DNA-binding domain (DBD) in metazoan genomes yet many influences that govern its base recognition remain poorly understood. This seemingly simplistic DBD recognizes a 3-4 base target using residues on the amino-terminus of its alpha helix. The stability of this small domain (typically 23 amino acids) is provided by the two cysteines and two histidines that coordinate a zinc ion as well as a small number of hydrophobic residues that pack into the core of the domain. These stabilizing residues can be thought of as structural as mutations at these positions lead to misfolding and loss of function. The remaining positions are somewhat flexible as a wide range of amino acids can be found at the nonstructural positions of the domain in nature. Screens of natural ZFs, as well as synthetic proteins that only assay residues on the helix, establish that examples of individual domains exist that are able to recognize any 3-base target. These results demonstrate that while any nonstructural residues within a single ZF may have subtle influences on specificity and affinity, recognition of any 3-base sequence can be as simple as 3-6 contacts provided by the residues on the helix. Nevertheless, models of ZF specificity struggle to accurately predict the target preference of ZF transcription factors (TFs), or even which ZFs of the protein engage the DNA. This failure implies that influences beyond the helix have been absent or underappreciated in our understanding of the domain. In this regard, genome editing has revolutionized how we do research today and offers enormous therapeutic potential. This revolution is largely because of the fast and robust development of CRISPR-Cas systems and the simplicity of their application. This technique offers unprecedented ease of use that has empowered labs across biological disciplines to create directed knockouts and knock-ins for nearly any model organism or cell type, opening the door to research that just a decade ago was reserved for yeast and mouse research. In addition, the fast development of these systems has created many alternative applications that harness their targeting power, such as epigenetic editing by expressing the dCas9 protein as a fusion to activation or repression domains. However, many of these tools and their applications were first developed by utilizing engineered Cys₂His₂ ZFs in the decades that preceded the discovery and development of CRISPR as an editing tool, but the difficulty of engineering the ZF domain and its inconsistent efficacy limited its application to a handful of highly skilled labs. Nevertheless, the ZF domain would offer advantages over alternative platforms if a new procedure was developed to routinely produce ZFs with high specificity and activity. For example, ZFs are significantly smaller than the spCas9 protein or an array of TALE domains, expanding the delivery strategies that might be employed. In addition, ZFs are the most common DNA-binding domain utilized by human transcription factors (TFs), representing over 7000 domains in the human proteome. Thus, their immunogenic potential is substantially lower than the non-human targeting domains Cas9, TALEs, and meganucleases.

As discussed above, the advantages of the ZF domain are understood and led to multiple attempts to engineer ZF arrays, yet none of the publicly available approaches have consistently produced ZFs able to bind any sequence with high efficacy. Thus, there is an ongoing and unmet need for improved compositions and methods that can used as an improved predictive model of ZF specificity. The present disclosure is pertinent to this need.

### BRIEF SUMMARY

The present disclosure provides methods that provide for determining amino acid sequences from a plurality of zinc fingers that bind to specific DNA substrates in a DNA sequence and zinc finger dependent manner. The binding of at least some of the zinc fingers in the plurality is determined by expression of a selectable marker and optionally a detectable marker. The method generally comprises the following steps:

i) providing DNA substrates that are operably linked to expression of the selectable marker, wherein each DNA substrate includes a segment comprising a DNA sequence configured to detect binding of a contiguous polypeptide comprising three distinct zinc fingers that are (F1 [domain 0]), F2 [domain 1]) and F3 [domain 2]), respectively, and wherein the F1 [domain 0], the F2 [domain 1] and the F3 [domain 2] are optionally in an N-terminal to C-terminal orientation;

ii) The plurality of zinc fingers are expressed in a series of in vivo assays within cells that comprise the DNA substrates. In each assay the F1 [domain 0] comprises the same amino acid sequence. In each assay, the F2 [domain 1] comprises at least one amino acid difference relative to domain 1 of other zinc fingers in other assays in the series. In embodiments, the amino acid difference is at position 6 of the F2 [domain 1]. F1 [domain 0] and F2 [domain 1] comprise a functional pair in each assay comprise known sequences. In each assay one or more of positions −1, 1, 2, 3, 5 and 6 of each F3 [domain 2] α-helix are randomized.

Using this configuration, the method further comprises:

iii) selecting zinc fingers that promote expression of the selectable marker; and, iv) determining the sequence of the zinc fingers that promote expression of the one or more markers to identify amino acids that promote the expression. The identified amino acids that promote the expression are included in at least the F3 [domain 2]._In embodiments, more than one, or all of positions −1, 1, 2, 3, 5 and 6 of each F3 [domain 2] α-helix are randomized. In embodiments, amino acid differences for each series of assays is at position 6 of the F2 [domain 1].

In embodiments, each assay in the series of assays comprises at least 64 million distinct F3 [domain 2] α-helices. In embodiments, a series of at least four assays are performed. In embodiments, 4-12 assays or are performed.

In embodiments, the segment of the DNA substrate configured to detect the binding in each assay in the series comprises at least one variable segment. The variable segment comprises three base pairs (bp) targets for use in determining the binding. The variable segments can comprise up to 64 different distinct 3 bp segments in each assay.

In embodiments, the series of assays is such that 64 independent selections can identify zinc fingers comprising a helices that may be able to interact with each of the 64 distinct 3 bp targets, and sufficient selections are performed such that at least 16 billion unique zinc figure-DNA substrate interactions may be present and analyzed for expression of a marker. In an embodiment 384 selections are made.

### BRIEF DESCRIPTION OF THE FIGURES

FIG. 1. Overview of interface-focused zinc finger screens. A. Structure of adjacent ZF domains showing their close proximity. Helical position 6 of domain 1 and position −1 of domain 2 are outlined. B. Cartoon of adjacent finger interactions with the DNA. The six helical positions of the two domains are shown as circles with the common contacts made by positions −1, 2, 3, and 6 shown with arrows. The overlap position, where both domains are able to specify the same base pair simultaneously, is outlined. C. Cartoon of the B1H selections. The 3-fingered protein is expressed as a C-terminal fusion to the omega subunit of RNA polymerase. For each library, ZF domain 2 is randomized at the six critical helical positions and screened for amino acid combinations able to specify each of the 64 possible “NNN” targets. Domains 0 and 1 bind to their known, preferred targets, fixed adjacent to the sequence where domain 2 will bind in each selection. The first base of the domain 1 binding site (the first A) represents the overlap base and the point of interface between the adjacent domains. Only helices in the library able to bind the target will recruit the polymerase, activate the reporter, and survive on selective media. D. (left) The helical residues of the 10 libraries sampled are shown. The library domain 2 contains all possible combinations of the six helical residues. Domain 1 is varied by library. The 6^{th} residue of domain 1 in each case is the side chain that will be exposed at the interface between domains 1 and 2. Domain 0 is the same in all libraries except library 1. In all cases we represent the 6 helical residues in the absence of position 4 as this position is hydrophobic, packs into the core of the domain, and does not contribute to specificity. In all of our libraries position 4 is a leucine. (right) There are 64 DNA targets for domain 2, one for every possible 3 bp sequence. These are all sampled in separate selections. The targets for domain 1 are shown with the overlap base in bold. E. PSSMs were calculated for all selections. The Information Content at the most conserved position of the helix is represented as a heat plot across all selections to provide a proxy of helical enrichment. F. Molecular dynamic simulations were performed on all domain 1 helices in their previously characterized contexts. The number of contacts made by domain 1 with the DNA are shown for each library as well as the structural fluctuation in the simulation (rmsd values). FIG. 1 sequences are RSDNRA (SEQ ID NO:4), RSDETR (SEQ ID NO:5), QLASTN (SEQ ID NO:76), DQSNTR (SEQ ID NO:77), FQSGIQ (SEQ ID NO:6), HKRNTD (SEQ ID NO:7), DQSALG (SEQ ID NO:8), TKQNTH (SEQ ID NO:9), QLATSY (SEQ ID NO:10), RNGNTR (SEQ ID NO:11), YQPNIN (SEQ ID NO:12).

FIG. 2. Construction of the Principal Component (pc) maps to represent the diversity of the zinc finger ‘helixome’. A. Flowchart illustrating the transformation of a 6 amino acid helix into 18 physical features and then reduced to two-dimensional coordinates pc1 and pc2 based on Principal Components Analysis. B. (left) Scatterplot representing all unique helices on the first and second principal component (pc1, pc2). (right) Zoomed region of the map shows helices with similar sequences (WXXXXR). C. left. 2D-density of helices. For each small squared box along pc1 (horizontal) or pc2 (vertical), helices are represented as sequence logos (right). Similarly, increasing the size of the box (bottom left) leads to more degenerate sequence logos. D. Projection of the scaled hydrogen-bond donor (+) and hydrogen-bond acceptor (−) features for each position in the helix. Positions −1 and 6 are circled in blue and red, respectively, with the position numbers labeled. E. Contour plots highlighting densities of helices filtered by their DNA target. All helices are split according to the target position 1 (Nnn) and shaded with 4 nuances for A, C, G and T. Similarly, helices binding the second target position (nNn) and the third one (nnN) are represented in a similar manner. FIG. 2 sequences are RSRSAK (SEQ ID NO:13), HGPSKK (SEQ ID NO:14), WASQSR (SEQ ID NO:15), WMNVKR (SEQ ID NO:16), WQAVAR (SEQ ID NO:17), WSQCRR (SEQ ID NO:18), WSSIIR (SEQ ID NO:19), WVSTTR (SEQ ID NO:20), WNHGRR (SEQ ID NO:21), CPNARH (SEQ ID NO:22), WNSALR (SEQ ID NO:23), WANQHR (SEQ ID NO:24), QSPAKY (SEQ ID NO:25), WPVRAR (SEQ ID NO:26), YMPHRR (SEQ ID NO:27), WCVSKR (SEQ ID NO:28), WFSGSR (SEQ ID NO:29), WSNLKR (SEQ ID NO:30), WCVTKR (SEQ ID NO:31), WPLRNR (SEQ ID NO:32), WDSSNR (SEQ ID NO:33), WSASDR (SEQ ID NO:34), HGPSHK (SEQ ID NO:35), WSSIR (SEQ ID NO:36), WVNSWR (SEQ ID NO:37), WVQNRR (SEQ ID NO:38), LPPTKH (SEQ ID NO:39), HSPPKK (SEQ ID NO:40) LSGGRK (SEQ ID NO:41), WNSASR (SEQ ID NO:42), WCNVKR (SEQ ID NO:43), WFQARR (SEQ ID NO:44), WSNLHR (SEQ ID NO:45), WVTTMR (SEQ ID NO:46), WKAGDR (SEQ ID NO:47), NAPSRK (SEQ ID NO:48), WHAGTR (SEQ ID NO:49), YKPNRR (SEQ ID NO:50).

FIG. 3. Visualizing selected zinc finger clusters on two-dimensional space. A. The MUSI defined clusters for AGT binding are color coded to show their positions on the PCA coordinates. While helices in these clusters generally localize to different regions of the map, helices in cluster 4 are found on two regions. Further detail shows that these subgroups, 4a and 4b, differ in the amino acids that appear to co-vary at the first and 4th position of the logo. B. In some cases the PCA maps do not sufficiently localize distinct clusters as shown in the sample CCT selection where cluster 1 and 2 overlap significantly. C. In other cases, subgroups of helices that are separated by the PCA coordinates are recognized as the same cluster by MUSI. D. However, by forcing MUSI to find more cluster provides finer resolution that in some, but not all cases, have been separated on the two-dimensions of the map.

FIG. 4. Visual comparison of specialized and general ZF populations by target sequence. A. PCA maps are shown for the CCA target selections for libraries 1(C), 3(A), 4, and 5. The general location of MUSI-defined clusters are indicated on the maps. (left) Three specialized clusters are shown as they were only recovered in a subset of libraries, noted below each logo. (right) The general cluster to bind CCA is shown. This cluster was found in 10 of the CCA screens, libraries noted below. B. Helices enriched in individual selections are plotted on the PCA coordinates. A comparison of the selection results for 4 of the most successful libraries are shown for the non-G binding targets CTT, TTC, and ATC (left) and three G-rich targets CGG, CTG, and GGG (right). (Bottom). A dot plot comparison of the 1-Hamming distance is provided for these same targets comparing libraries 1 thru 9. The darkness of the dot represents the similarity of the enriched populations with dark dots being more similar and light dots less similar. The spot is empty if the target selection failed for one or both of the libraries compared. C. Normalized hamming distance for all libraries across all targets listed from least similar (left) to most similar (right). The targets compared in B are underlined.

FIG. 5. General and specialized solutions for the GTG and CCG selections. A. PCA plots are shown for GTG (top) and all CCG (bottom) selections from all of highly successful libraries. Libraries 6 and 10 were omitted due to poor performance. A boxed region for all GTG selections shows the location of the RxxxxR cluster recovered in all selections. A boxed region in all CCG selections shows the location of a QxRYxx motif absent in 3 of the selections. Below, for each library the amino acid at the interface, position 6 of domain 1, is shown. The QxRYxx is absent in screens that have an arginine at this position. B. Schematic illustration (left) and molecular dynamics snapshot (right) of the hydrogen bonds between the arginine at position 2 of the QsRYtt domain 2 helix with the G* of the CCG* target. (top) The interactions in the library 2 context that positions an asparagine at the interface. (bottom) The interactions in the library 3 context that positions an arginine at the interface, demonstrating a non-canonical, competitive interaction between this arginine and G*. C. Frequency of hydrogen bond contacts in the Molecular Dynamics trajectories. (left) We distinguished single (S) and double (D) hydrogen bonds between G* and the arginine in the QsRYtt helix. Clear bars represent the library 2 context and shaded bars represent library 3. (right) We distinguished hydrogen bonds between G* and the asparagine at position 6 of domain 1 for library 2 from the arginine at position 6 of library 3. D. Variations of helix families for the four NCG targets. For all 4 targets the QxRYxx population is found in the library 2 selections but not libraries that place an arginine at the interface (library 9 shown) suggesting a common incompatibility of binding strategies across similar target sequences. E. Cartoon of adjacent finger interactions with the DNA showing the multiple potential contacts made by position 2 of the helix and the novel contacts suggested here (Domain 2 position 2—B3, Domain 1 position 6—B3).

FIG. 6. Zinc finger nuclease disruption of eGFP reporter. A. Schematic of a dimeric ZF nuclease bound to the DNA. A longer linker between ZF pairs allows a base to be skipped between targets and maintains independent of the 2-finger modules. B. Screen of 42 ZFNs that recognize sequences in the eGFP coding sequence. Pairs of ZFN monomers were nucleofected into a U20S reporter cell line in the presence of a control (tdTom) and sorted to recover populations more likely to have taken up both ZFN monomers. The mean fraction GFP negative cells are shown, ordered from the least active to most active nucleases. A Cas9 assay paired with a strong guide was carried out in parallel for all experiments to provide a common positive control. C. Five ZFNs with disparate activity levels were expanded from 8-finger ZFNs (4 ZFs per monomer, solid) to 12-finger ZFNs (6 ZFs per monomer, checkered). Mean GFP negative cells are shown for both 8 and 12-finger versions of these nucleases. D. The influence of non-specific affinity on nuclease activity. Phosphate contacts were reduced in the ZFs of the 8-finger (solid) and 12-finger (checkered) versions of the same ZFN. ZFNs with phosphate mutations are labeled as “mut”. Mean GFP negative cells are shown for the bulk populations.

FIG. 7. Zinc finger interface and common selection strategies. A. Cartoon of two adjacent fingers interacting with DNA. The six positions of the helix with base-specifying potential are shown. Position 4 is omitted as it is typically a hydrophobic residue that packs into the core of the domain. It is not randomized in any selection schemes. The interface and contacts are highlighted with an oval. B. Cartoon of a single finger selection approach where all the randomization is on one of the two fingers (PMID: 10077584, 18657511, 8303274). These were almost universally done with an arginine-guanine contact (highlighted) adjacent to the selected finger or, in one case, where the library was the N-terminal finger. On the randomized helix the letter's in bold and red (CFWY) were not coded for in the OPEN zinc finger libraries. C. Two versions of libraries that selected interface interactions are shown. Top. (PMID: 22543349) Many of the contacts were fixed with 5 positions incompletely randomized. The red and bold amino acids were not available in these libraries. Bottom. (PMID: 11433278) Another approach randomized more positions but used a very small subset of amino acids. Only available amino acids are listed. FIG. 7 sequences in the cartoons are RTEDSR (SEQ ID NO:63) and TDSR (SEQ ID NO:64). The FIG. 7 sequence shown repeated and vertically is ACDEVFGHIKLMNPQRSTVWY (SEQ ID NO:65).

FIG. 8. Libraries and interfaces tested. Table. All libraries screened in this disclosure are listed. The helical residues for the zinc finger adjacent to the library (domain 1) are shown for each library. The residue presented at the interface (underlined), the overlap base, and the biophysical category of this side chain is noted. Helical enrichment numbers and selection success is also listed. A. Cartoons are shown to depict what environment is presented to the selected zinc finger in each library with A overlaps on the left, C overlaps on the right, and G overlaps at the bottom. FIG. 8 sequences shown in the cartoons are ARNDSR (SEQ ID NO:51), NSTALQ (SEQ ID NO:52), RTNSQD (SEQ ID NO:53), QIGSQF (SEQ ID NO:54), NINPQY (SEQ ID NO:55), ARNDS (SEQ ID NO:56), DTNRKH (SEQ ID NO:57), GLASQD (SEQ ID NO:58), HTNQKT (SEQ ID NO:59), YSTALQ (SEQ ID NO:60), RTNSQD (SEQ ID NO:61), RTNGNR (SEQ ID NO:62). FIG. 8 sequences in the table are RSDNLRA (SEQ ID NO:66), QLATLSN (SEQ ID NO:67), DQSNLTR (SEQ ID NO:68), FQSGLIQ (SEQ ID NO:69), HKRNLTD (SEQ ID NO:70), DQSALLG (SEQ ID NO:71), TKQNLTH (SEQ ID NO:72), QLATLSY (SEQ ID NO:73), RNGNLTR (SEQ ID NO:74), YQPNLIN (SEQ ID NO:75).

FIG. 9. 1-Hamming distance dot plot comparison of libraries by target sequence. Here we compare the similarity of all successful selections for the screens of the primary libraries 1 thru 9 for all 64 triplets. As the plot is 1-Hamming distance, the darker the dot, the more similar the selections. An empty space indicates that the selection for one or both of the libraries failed and therefore no comparison can be made. All plots are on a scale of 0.4 to 1 so that comparisons can be made between plots. Gnn (vertical) and nnG (horizontal) targets are boxed to highlight how similar these selections.

FIG. 10. Global Hamming distance comparisons for libraries that present different overlap bases at the interface. A. Hamming distance comparison across all successful selections for library 1(A)-top, 2-middle, and 4-bottom, with the remaining libraries that were successful across most target selections. Libraries 6 and 10 were omitted because of their poor performance. A-overlap libraries are to the left and C-overlap libraries to the right. Libraries 1(A), 2, and 4 all bind adenine at the overlap and for the most part they are more similar to other A-overlap libraries than they are to C-overlap libraries. B. Libraries 1 and 3 are able to bind A or C and A or G, respectively, at the overlap. A comparison of these libraries using A at the overlap demonstrated that the same library with a different base at the overlap is approximately as similar as the comparison to other A-overlap selections. C. A comparison of library 9, that uses an arginine-guanine contact at the interface, is significantly more similar to the only other library screened that also placed an arginine-guanine contact at the overlap, than compared to any other library screened.

FIG. 11. Shown in A., We retrieved all specific contacts between DNA bases and protein side chains from 1247 structures of DNA:protein complexes in the Protein Data Bank. We considered only specific contacts that involve the bases and atoms A N6, A N7, C C5, C N4, G N7, G O6, T C7 and T O4. The contact was considered if the distance between two neighboring atoms is inferior to 3.5 Å. Hydrogens where not considered. In average, 4.7 contacts are found per Protein:DNA complex, including the common bifurcated hydrogen bonds. B. We ordered the amino-acid bases pairs according to their occurrence in the structures. It is noteworthy that Arginine:Guanine are the most prevalent pairs in the structures and in our Bacterial 1-Hybrid experiments. It is likely that this pair contributes to a high affinity complex when two hydrogen bonds form between the Guanine acceptor groups O6 and N7 with the Arginine NH1 and NH2. C. We extracted all these pairs and align them on the Guanine base. The resulting distribution of Arginine position shows that the double hydrogen bond is found in the majority of the pairs (72%) where the Guanidine base and the Arginine Guanidium groups are observed on a same plan.

FIG. 12. Comparison of PCA maps within and between library selections. A. The GAN selections for Library 1 are shown. A common region in each map is boxed in orange to show the dominant family in all selections are roughly found in the same region of the map. These helices mostly specify GAn. However, helices enriched in other regions of the map are more dependent on the 3rd base of specificity. B. A comparison of the AAC selections for libraries 1, 3, 7, and 8. A common region is boxed in red to show how this family of AAC-binding helices is differentially enriched based on the library context.

FIG. 13. Two-finger selection workflow. A. For a given 6 bp target, the DNA recovered from each of the two 3 bp half site were pooled from successful primary selections. In the example shown, the pools from all successful ATG selections were combined and the pools from all successful CAT selections were combined. These pre-selected pools were used as templates for PCR that would assemble the ZFs as adjacent pairs after a 2nd round of PCR. B. These pre-selected 2-finger pools were then cloned into our expression vector that will express the 2-finger library as a fusion between the omega activation domain and two constant ZFs with known specificity. The known ZFs interact with a fixed binding site and thus position the library fingers to bind the desired target, in this case ATG-CAT. Compatible pairs survive the selection. C. Approximately 1 million cells transformed with the ZF library and the complementary target-reporter are grown in 1 ml selective media which can be scaled to a 96-well format. When the cultures have grown to saturation the cells are recovered, DNA harvested, and sequenced.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. The disclosure is not intended to be bound by any particular theory described herein. The disclosures of all references described in this disclosure are incorporated herein by reference.

Representative compositions and methods are provided. The disclosure includes all compositions and steps as described herein and as shown in the accompanying figures. The disclosure includes the proviso that any single or combination of reagents and steps may be excluded. Some or all the steps may be performed sequentially, although concurrent performance of steps is not necessarily excluded from the disclosure. The disclosure includes all compositions of matter formed during performance of the described method. The disclosure includes all expression vectors and combinations of expression vectors used to produce, screen and identify zinc fingers as further described herein. The disclosure relates in part to performance of a series of assays. The zinc fingers encoded by expression vectors in the assays, and the expression vectors themselves, may be considered libraries.

The disclosure includes all described methods of measuring and displaying results obtained from the described assays. The disclosure includes use of Hamming distance to obtain any described measurement. Hamming distance determination is known in the art, and generally involves measuring the edit distance between two sequences.

The term “zinc finger” is abbreviated from time to time in this disclosure as “ZF.” The term “finger” also refers to a zinc finger. The zinc fingers referred to in this disclosure generally comprise Cys₂His₂ zinc fingers, but other types of zinc fingers are not necessarily excluded.

ZFs used and identified in assays as further described herein are comprised by contiguous polypeptides. The contiguous polypeptide may be described in an N-terminal to C-terminal orientation due to the anti-parallel DNA binding that characterizes ZF-DNA binding. Each contiguous polypeptide generally comprises a three ZF series. Contiguous polypeptides comprising only two ZF proteins may also be used, as further described in the Examples. Each finger can be considered F1, F2, and F3, which are also referred to herein as domain 0, domain 1 and domain 2, respectively. In embodiments, two ZF's may be separated from one another in a contiguous polypeptide by an intervening linker segment. In general, a suitable linker between two zinc fingers is 8 or fewer amino acids long, and may be present between the last His of the N-terminal finger and the first Cys of the C-terminal finger, to consider them part of the same array. As a non-limiting example, the canonical zinc finger linker TGEKPFA (SEQ ID NO:3), or derivatives of this linker, are found in the majority natural zinc finger proteins between the 2nd His of an N-terminal finger and the first Cys of the following finger. In one embodiment, a linker is used to separate each ZF pair and skips a base between their targets (PMID: 30850604).

The present disclosure provides compositions and methods for use in improved analysis of each factor that impacts ZF-DNA interaction in context, and how they influence the ZF-DNA engagement, both individually and combinatorially.

In certain approaches, the present disclosure provides a method of determining amino acid sequences from a plurality of zinc fingers that bind to specific DNA substrates in a context dependent manner, wherein the binding of at least some of the zinc fingers in the plurality is determined by expression of a selectable marker and optionally a detectable marker.

In embodiments, interacting zinc finger combinations are selected by binding to DNA, such as a 3 base pair segment, which in turn drives expression of a detectable marker, or a selectable marker, or a combination thereof. In a non-limiting embodiment, the zinc fingers are selected using a series of bacterial one-hybrid assays, but alternative assays may be used, including but not necessarily limited to yeast-based assays, and assays performed in mammalian cells, including but not necessarily limited to human cells. Thus, any assay that provides a readout of zinc finger binding can be adapted for use in the described methods. In embodiments, bacterial one-hybrid assays are performed using the zinc finger and

DNA substrates as further described herein and by adapting bacterial one-hybrid assay described in Persikov, et al., (2015). A systematic survey of the Cys2His2 zinc finger DNA-binding landscape. Nucleic acids research, 43(3), 1965-1984, and Noyes M. B. Analysis of specific protein-DNA interactions by bacterial one-hybrid assay. Methods Mol. Biol. 2012; 786:79-95 from which the disclosures of bacterial one-hybrid protein selections are incorporated herein by reference.

The described assays can be adapted to use different selectable or detectable markers, and combinations thereof. Positive and negative selection, and combinations thereof can be used. The specific markers used are not particularly limited. In embodiments, the selectable marker comprises a gene that encodes a protein that produces or participates in production of a substance that is required for an organism to remain viable, which may be related to the components of a culture medium, non-limiting embodiments of such genes comprising HIS3 and URA3, or any other auxotrophic marker. In embodiments, the selectable marker may alternatively be an antibiotic resistance gene, such as a gene whose protein product provides resistance to, for example ampicillin, kanamycin, chloramphenicol, tetracycline or triclosan. In addition, URA3 can be used for negative selection in the presence of 5-fluoroorotic acid (SFOA).

The detectable marker may be any marker that produces a detectable signal, non-limiting embodiments of which include green fluorescent protein (GFP), enhanced GFP, mCherry, mTAGBFP2, mPlum, YFP, mPapaya, mStrawberry, blue fluorescent protein (BFP), Sirius, and the like. In embodiments, the detectable labels produce a signal that comprises UV light (<380 nm), visible light (380-740 nm) or far red (>740 nm). Colorimetric assays markers may also be used, such as a β-galactosidase assay.

The selection of particular combinations of interacting zinc fingers can be adjusted to require, for example, a threshold level of expression of the selectable and/or detectable markers, which may be correlated with affinity of the zinc fingers for a particular DNA sequence.

The mutation and randomization of zinc fingers can be performed using any suitable techniques, such as site directed mutagenesis. The sequence of zinc fingers can be determined by sequencing of plasmids or other expression vectors that are used to express the zinc fingers which selected by using the pertinent host cells and selection approaches described above and further herein. The disclosure includes identifying interacting zinc fingers using the describe methods, producing the zinc fingers by any suitable protein expression technique, and using the identified zinc fingers and combinations thereof for any purpose.

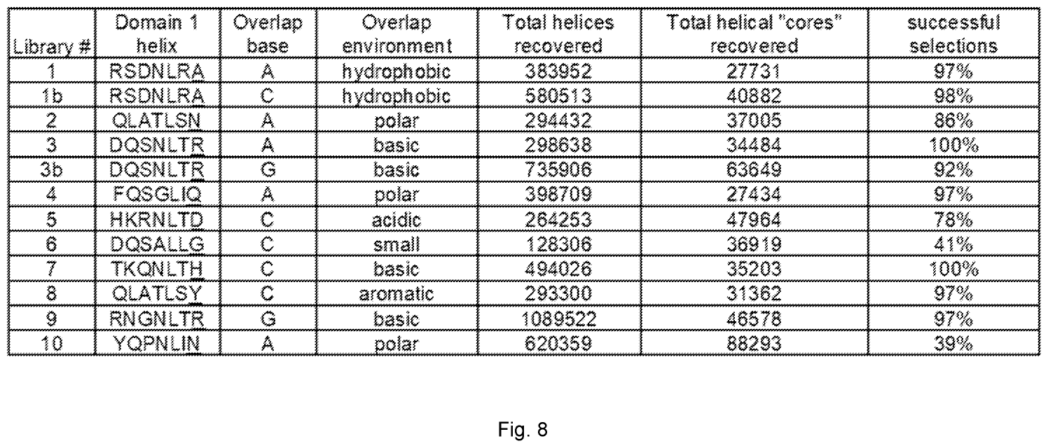

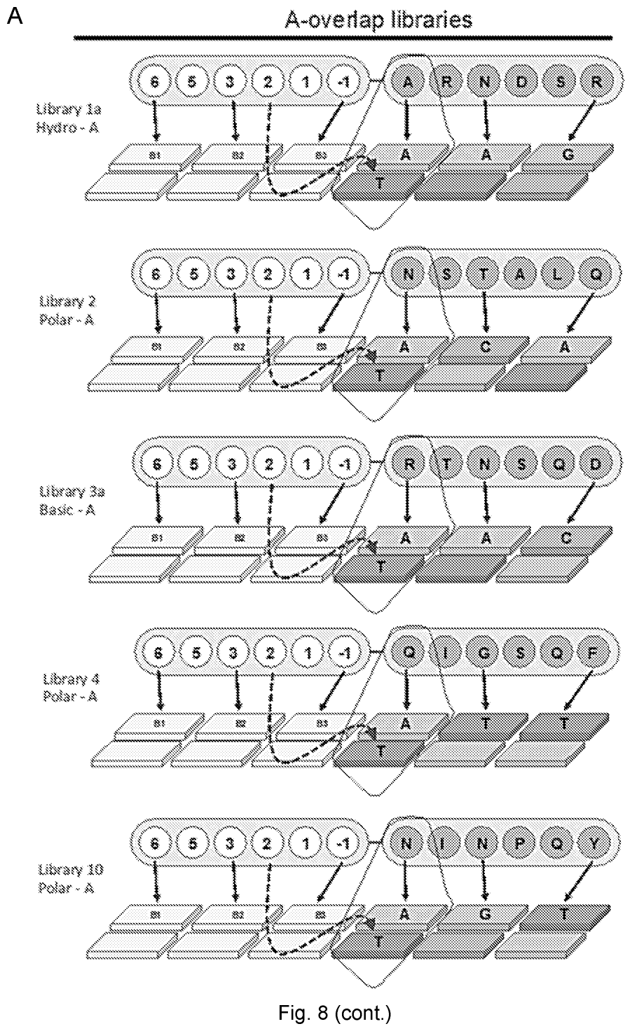

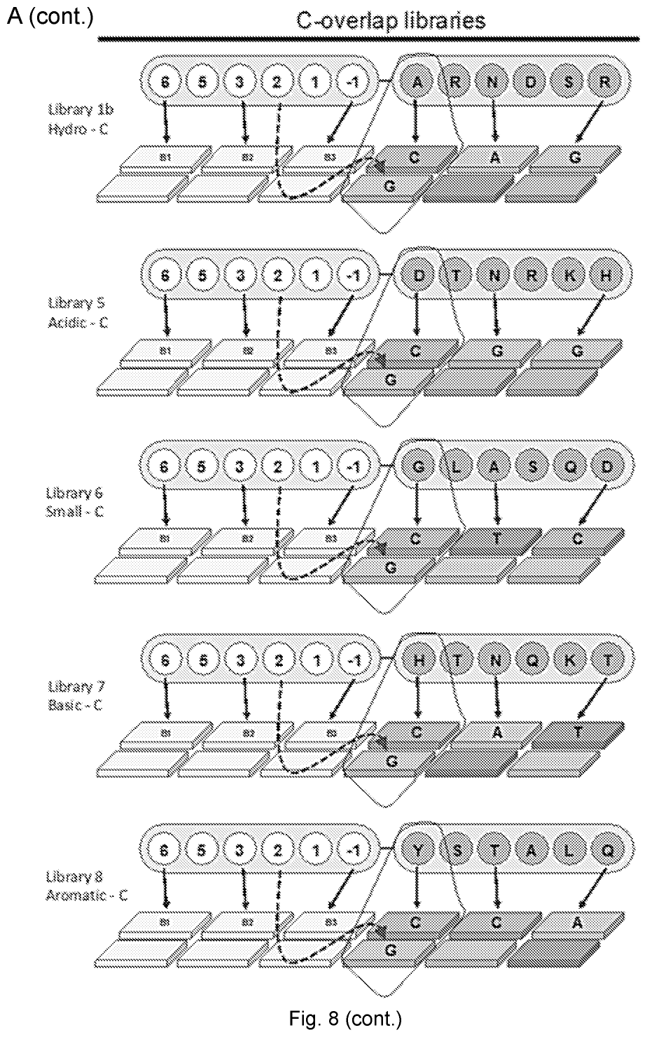

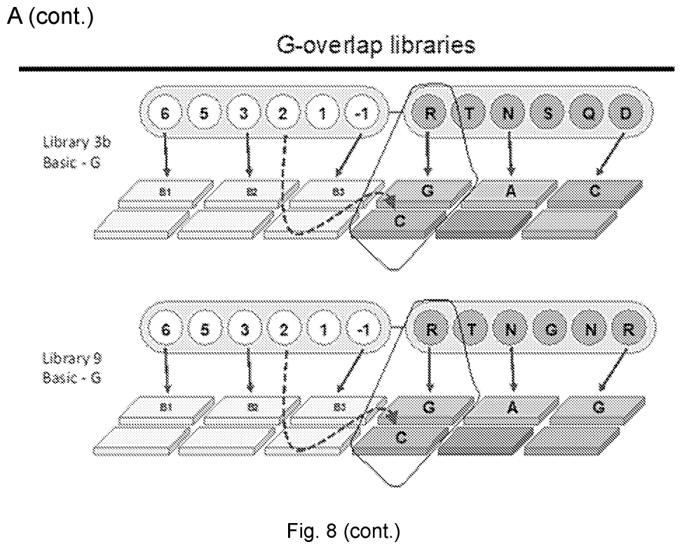

In embodiments, the disclosure provides for selection and identification of zinc fingers in a context dependent manner. By “context dependent” it is meant that the described assays take into account the influence of adjacent fingers on one another, and particularly the relationship of F2 and F3. Accordingly, the described assays provide for the first time identification of the amino acids in F3 that promote expression of a marker due to the F3 interaction with the DNA that takes into account influence of its interaction with F2. While the described assays include analysis of ZFs that comprise randomized α-helix amino acids in positions −1, 1, 2, 3, 5 and 6 of each F3 [domain 2], without intending to be bound by any particular theory, it is considered that position 6, and potentially position 5, of F2 have increased influence on F3 DNA binding due to their close proximity when bound to DNA, relative to the other stated positions, such influence not having been previously analyzed in the same depth as in the present disclosure. (See FIG. 8, for example.) Thus, in embodiments, the disclosure provides for comprehensive analysis of the proximal interaction(s) presented by F2 that influences F3's engagement with a particular 3 base pair (bp) DNA sequence. Accordingly, in F2's in each of the described assays, at least one amino acid is different in each F2, optionally at position 6, relative to F2's in at least some other assays in the series.

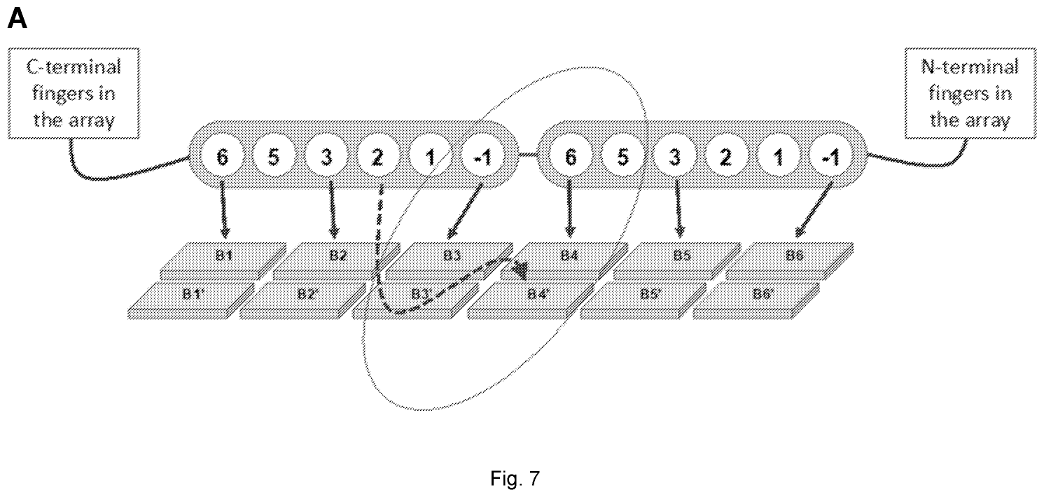

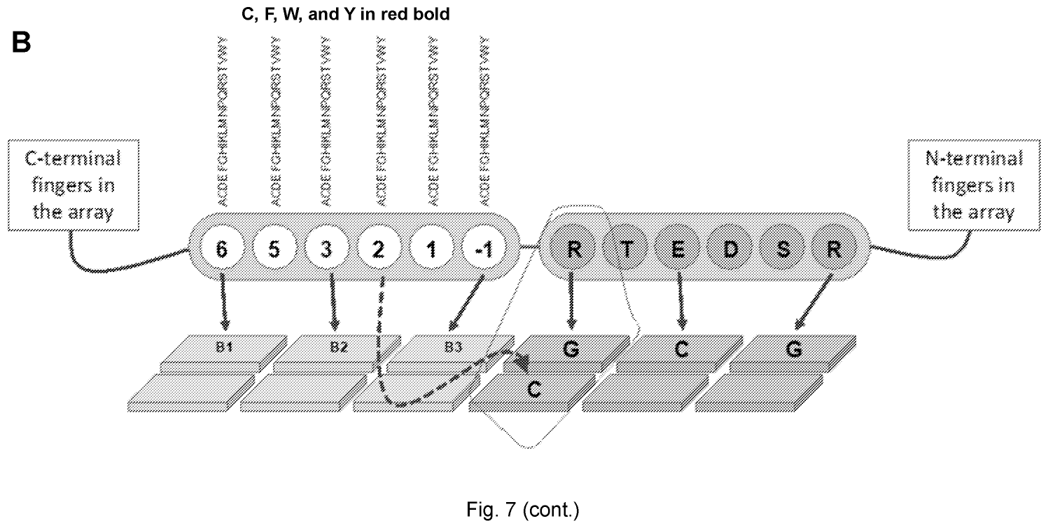

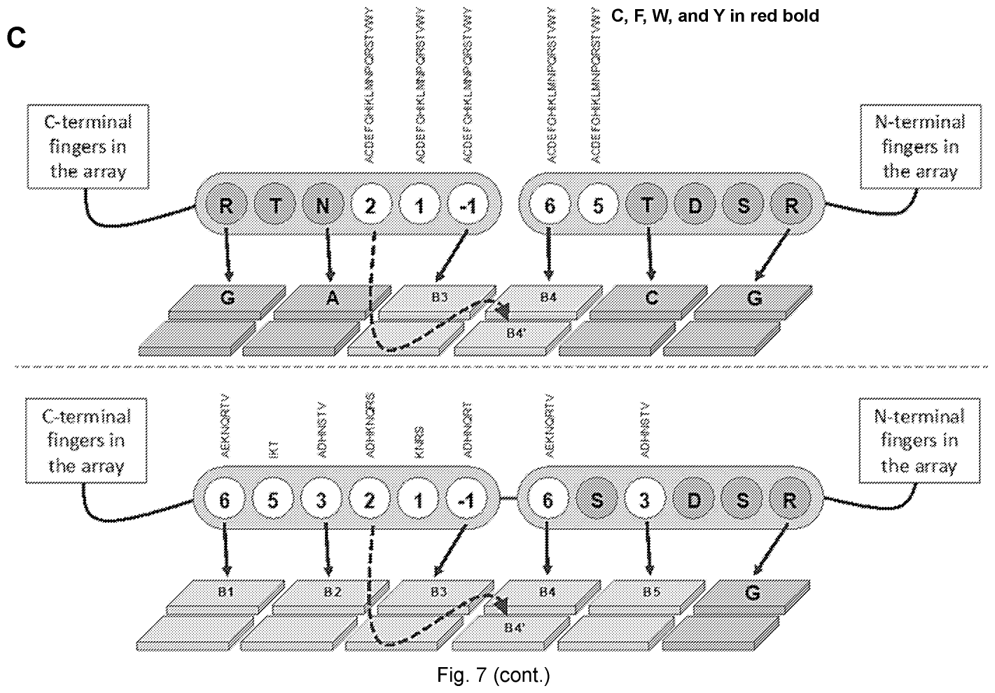

The disclosure includes use of F2's that, in at least some of the assays, do not include an Arginine in position 6 of F2, which is one aspect that differentiates the present disclosure from previous approaches. Accordingly, the presently provided approach (as illustrated at least by comparison of FIG. 7B and FIG. 100) permits focusing selections on the influence at the interface of F2 and F3, and produces significantly different data than what was generated using previously available methods.

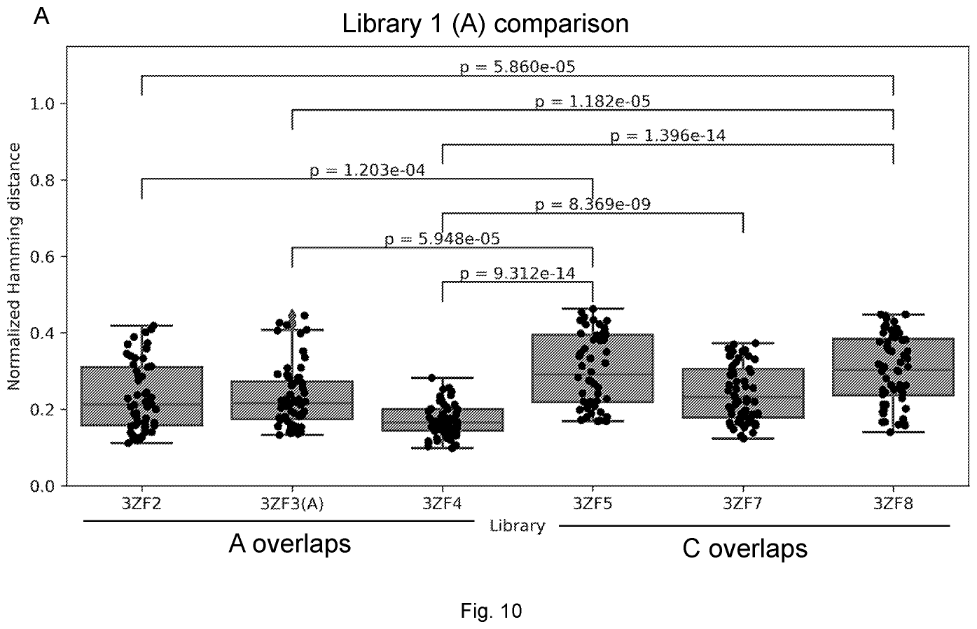

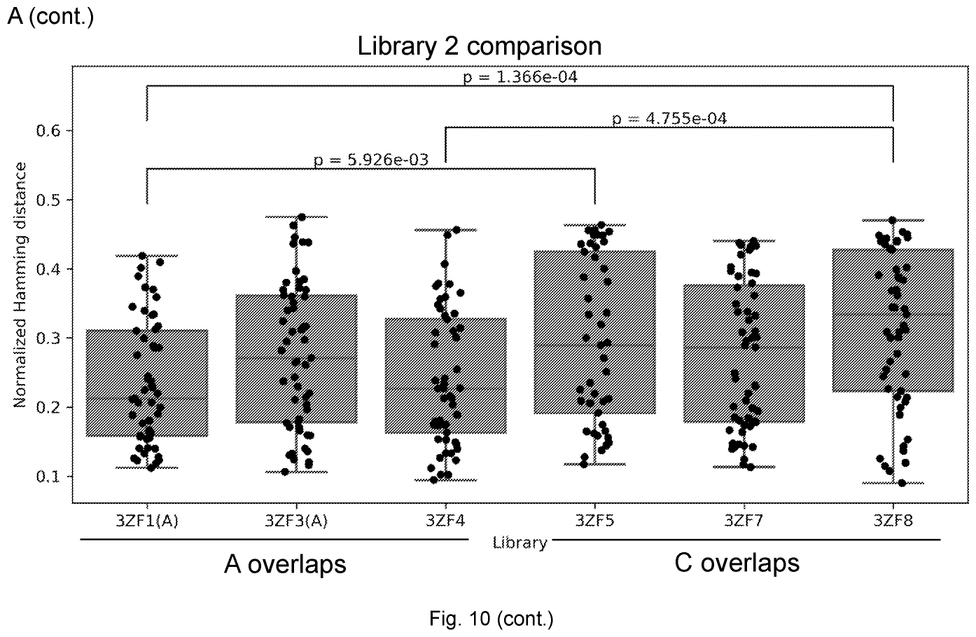

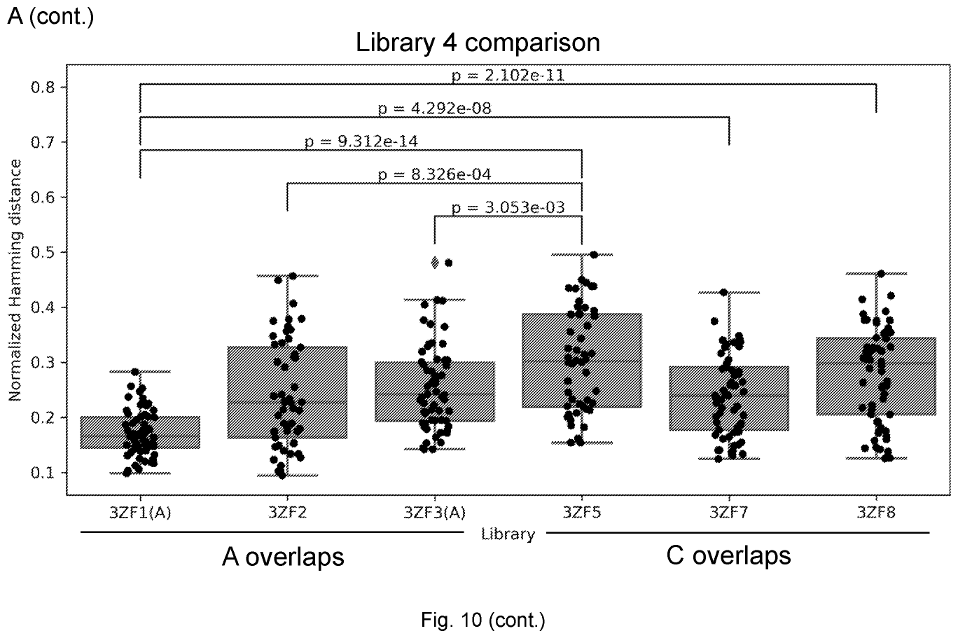

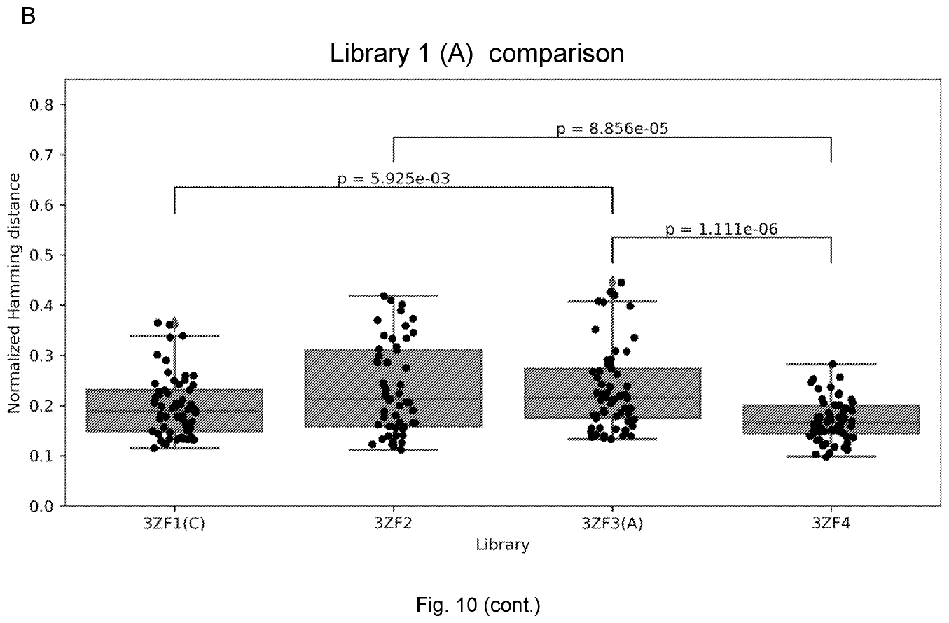

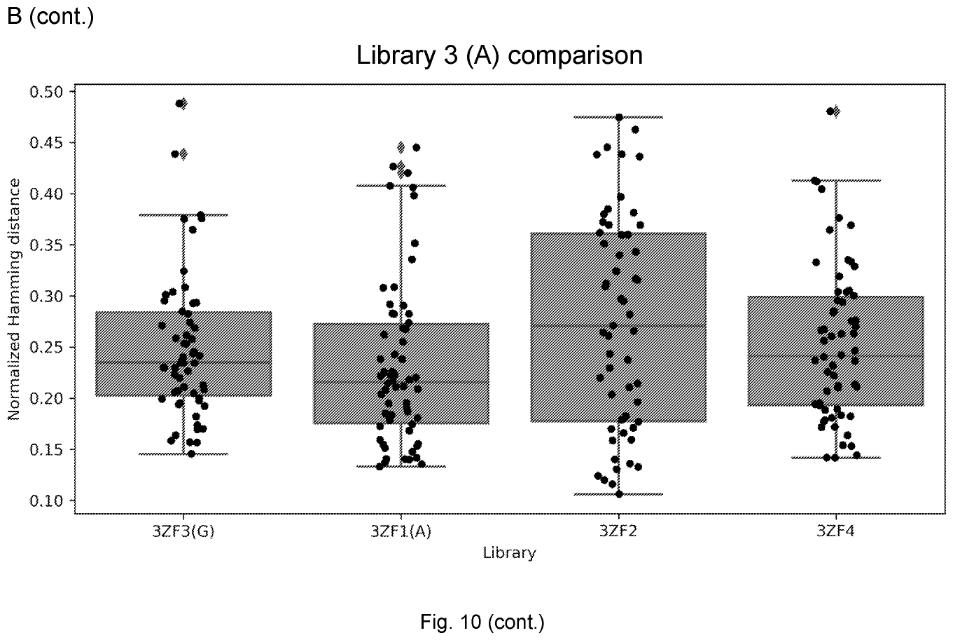

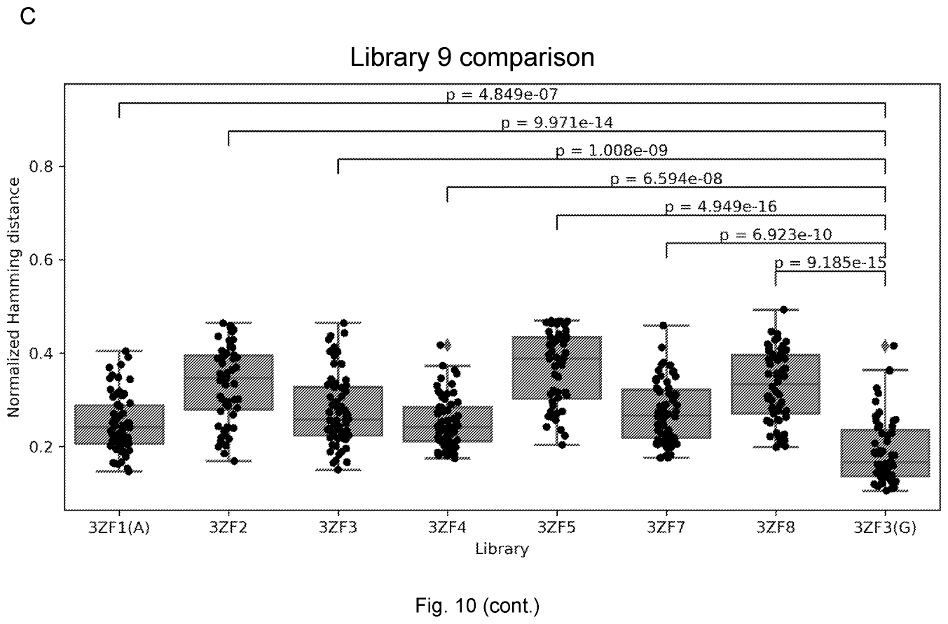

Further differentiation from previous approaches is illustrated at least by FIG. 7 which shows that previous approaches used one or more fixed Arginine-guanine contacts in combination with incomplete randomization of F3 [domain 2]. Further, the dependence on arginine-guanine contacts restricts prior approaches to binding to G-rich DNA substrates. Thus, the disclosure provides libraries that positions the library finger (e.g., F3 or domain 2), the 3^{rd} in an array, immediately adjacent to two ZFs that have known, fixed interactions with their target sequences. FIG. 10 provides a representative depiction of this configuration, and shows that the library finger is positioned directly over the 3 bp target that is varied in each target selection. Accordingly, in certain embodiments, each library differs from the others only by the fixed interaction between the middle finger and the DNA, thereby providing a novel interface environment between the middle and library fingers.

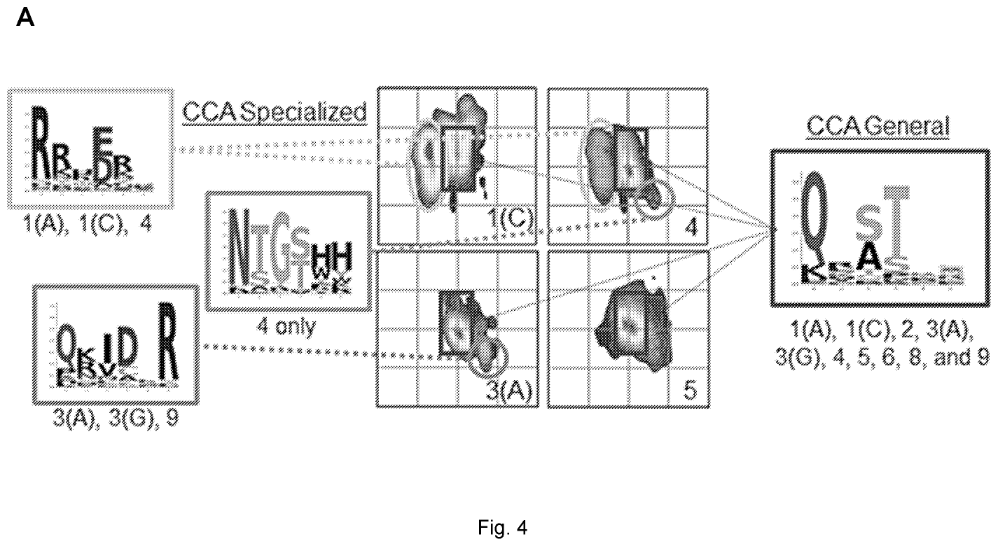

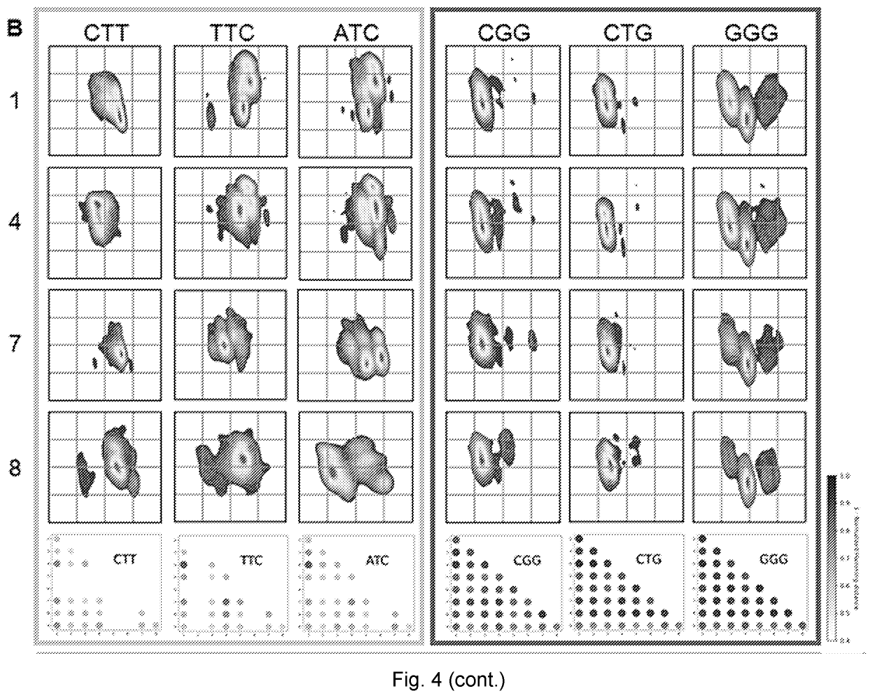

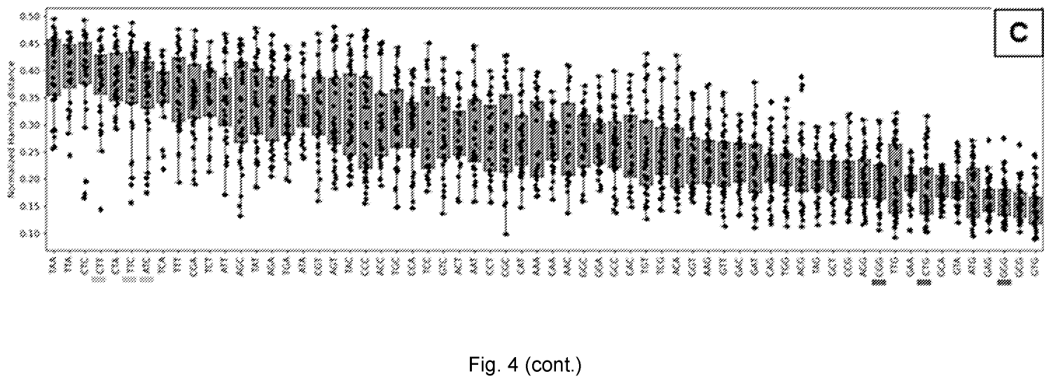

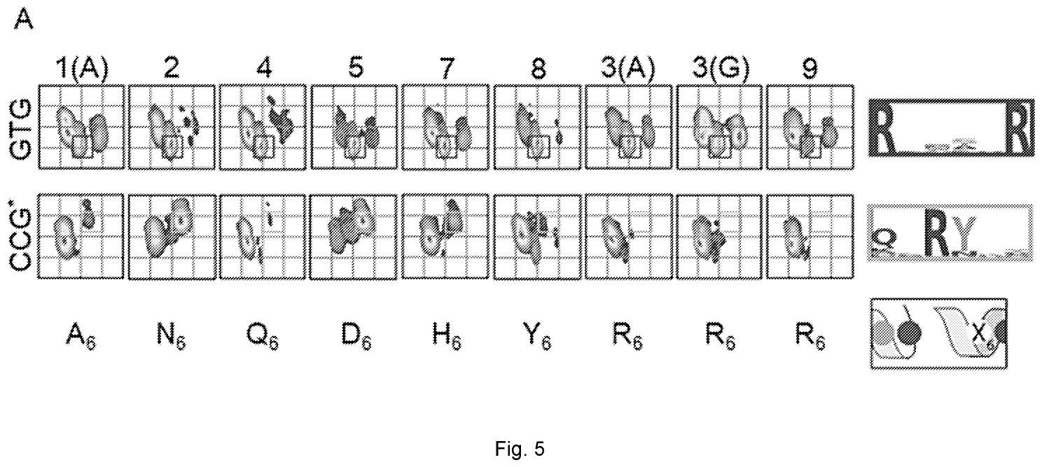

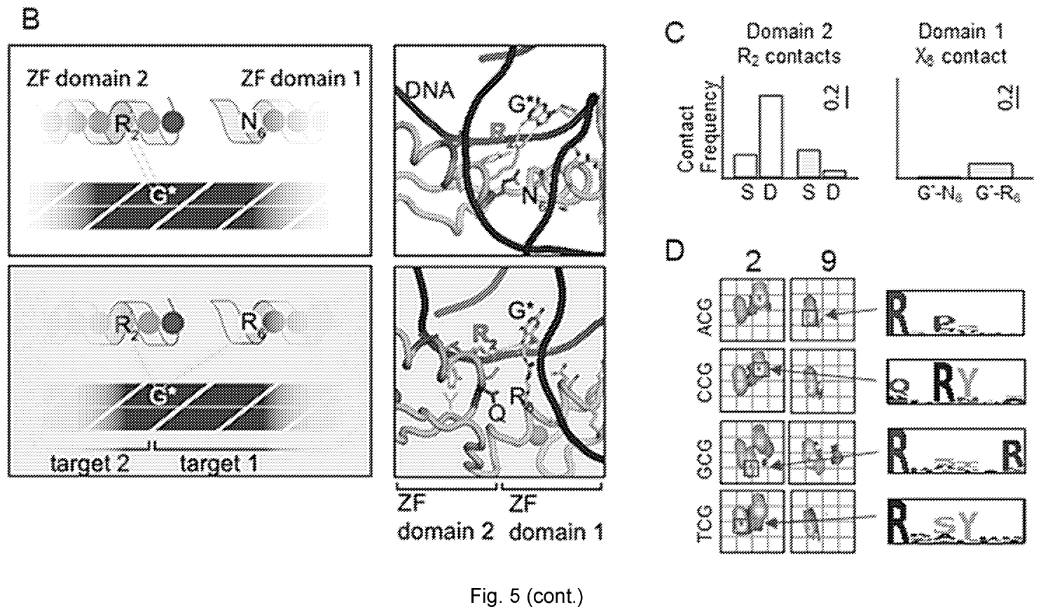

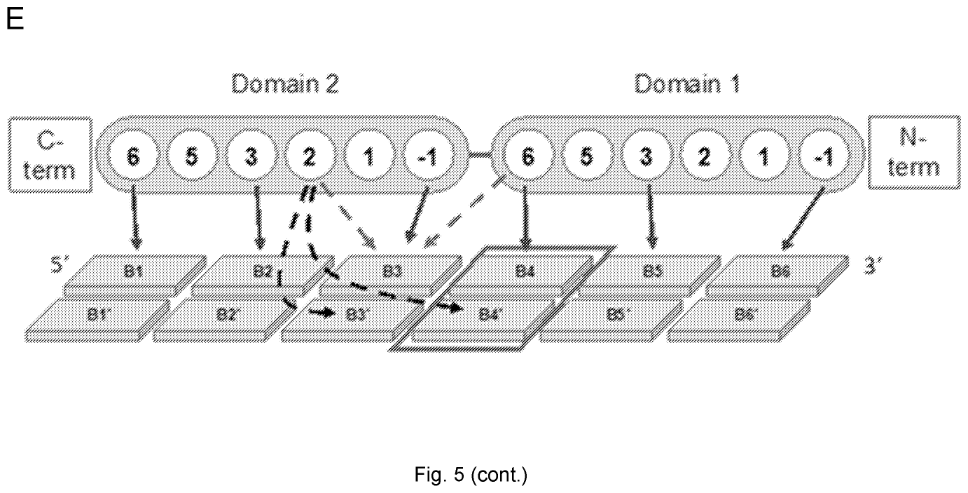

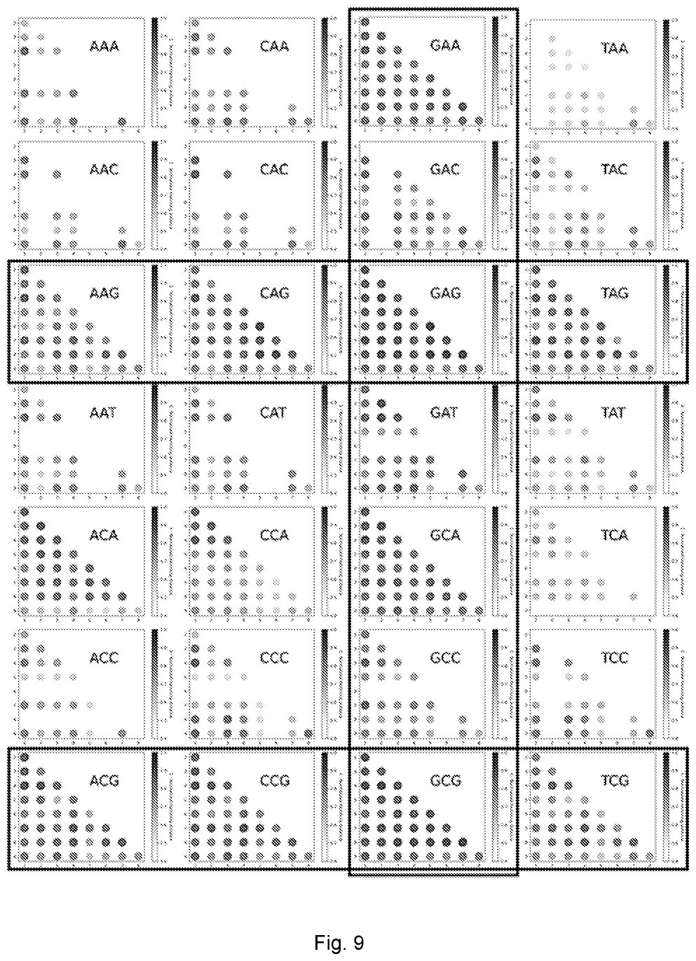

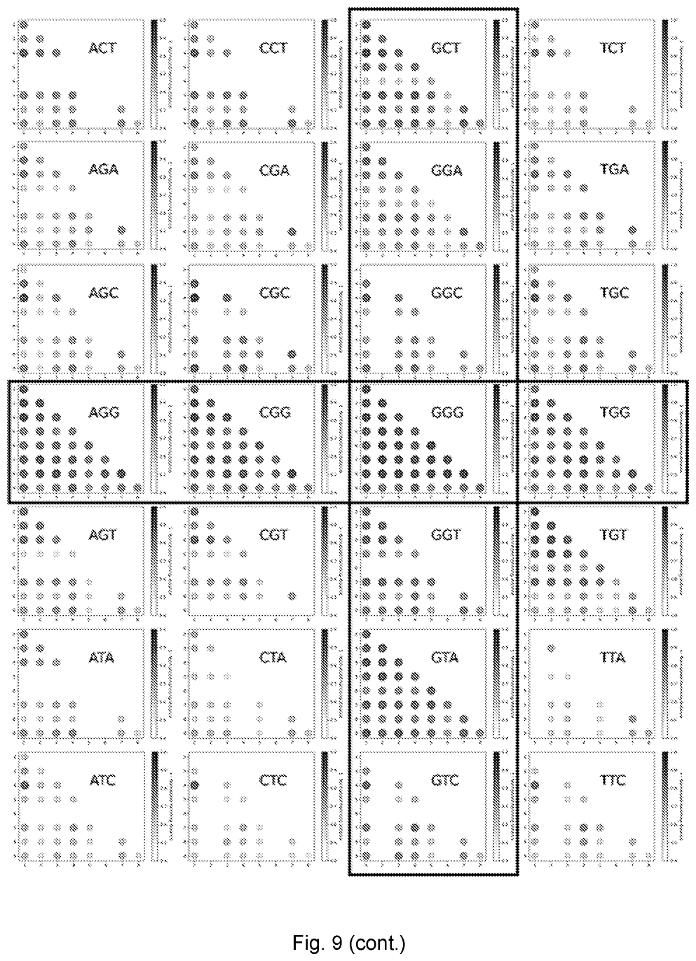

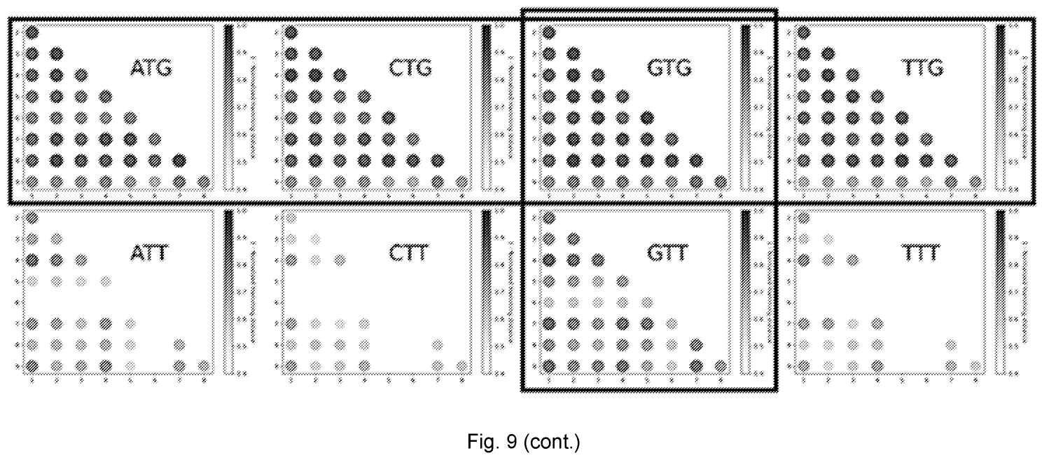

In more detail, as shown in FIG. 9 and FIG. 4C, the disclosure illustrates that G-rich binding is the least influenced by the adjacent finger (F2). However, A/C/T DNA substrate binding are greatly affected by F2-context. The present disclosure demonstrates that there are many more different solutions to bind such sequences in one library versus another, and that these fingers are highly influenced by the F2 context. As a specific and non-limiting illustration of this relationship between F3 and F2, FIG. 5 demonstrates a helical motif of QxRYxx binding CCG that is never selected when an arginine is at position 6 of F2, but the particular ZF analyzed in that assay does bind CCG when an Arginine is not at position 6 of F2. Thus, and without intending to be bound by any particular theory, it is considered that at least in part by providing F2's that do not have Arginine in position 6 of F2 in at least some of the assays that are performed in the series of described assays, the present approach is superior in identifying ZFs that bind to any particular 3 bp sequence including those that may be context dependent.

As also described further herein and by way of the figures, this superior approach is achieved in part by using assays that include F3's [domain 2's] having positions −1, 1, 2, 3, 5 and 6 of each F3 α-helix randomized, but in the context of an F2 [domain 1] wherein at least the position 6 amino acid in F2 and its interaction with the DNA, e.g., different DNA substrates, is different in each assay, and wherein F1 [domain 0] is fixed throughout an assay series, and as stated above, in at least some F2's position 6 is not an Arginine. In embodiments, at least some assays include adenine or cytosine at this overlap position, in addition to the common arginine-guanine contact in other assays.

By using this approach, the F3 is placed in a context where domain 1 and domain 2 form a functional pair. By “functional pair” it is mean that domain 1 and domain 2 will function together to bind a particular 3 base pair DNA segment when provided with a suitable domain 3, thereby providing a selectable combination of zinc fingers. Thus, the amino sequence of domain 0 and domain 1 of each ZF is known in each assay, domain 0 and domain 1 are known to be compatible with each other, and each domain 2 [F3] α-helix is randomized to determine the sequence of which F3 interacts with the functional pair comprised by fixed domain 0 [F1] and domain 1 [F2] having at least position 6 changed in individual assays.

Thus, in various aspects the disclosure provides methods that provide for determining amino acid sequences from a plurality of zinc fingers that bind to specific DNA substrates in a DNA sequence and zinc finger dependent manner. The binding of at least some of the zinc fingers in the plurality is determined by expression of a selectable marker and optionally a detectable marker. The method generally comprises the following steps:

i) providing DNA substrates that are operably linked to expression of the selectable marker, wherein each DNA substrate includes a segment comprising a DNA sequence configured to detect binding of a contiguous polypeptide comprising three distinct zinc fingers that are (F1 [domain 0]), F2 [domain 1]) and F3 [domain 2]), respectively, and wherein the F1 [domain 0], the F2 [domain 1] and the F3 [domain 2] are optionally in an N-terminal to C-terminal orientation;

ii) The plurality of zinc fingers are expressed in a series of in vivo assays within cells that comprise the DNA substrates. In each assay the F1 [domain 0] comprises the same amino acid sequence. In each assay, the F2 [domain 1] comprises at least one amino acid difference relative to domain 1 of other zinc fingers in other assays in the series. In embodiments, the amino acid difference is at position 6 of the F2 [domain 1]. F1 [domain 0] and F2 [domain 1] comprise a functional pair in each assay comprise known sequences. In each assay one or more of positions −1, 1, 2, 3, 5 and 6 of each F3 [domain 2] α-helix are randomized.

Using this configuration, the method further comprises:

iii) selecting zinc fingers that promote expression of the selectable marker; and, iv) determining the sequence of the zinc fingers that promote expression of the one or more markers to identify amino acids that promote the expression. The identified amino acids that promote the expression are included in at least the F3 [domain 2]. In embodiments, more than one, or all of positions −1, 1, 2, 3, 5 and 6 of each F3 [domain 2] α-helix are randomized. In embodiments, amino acid differences for each series of assays is at position 6 of the F2 [domain 1].

In embodiments, each assay in the series of assays comprises at least 64 million distinct F3 [domain 2] α-helices. In embodiments, a series of at least four assays are performed. In embodiments, 4-12 assays or are performed.

In embodiments, the segment of the DNA substrate configured to detect the binding in each assay in the series comprises at least one variable segment. The variable segment comprises three base pairs (bp) targets for use in determining the binding. The variable segments can comprise up to 64 different distinct 3 bp segments in each assay.

In embodiments, the series of assays is such that 64 independent selections can identify zinc fingers comprising a helices that may be able to interact with each of the 64 distinct 3 bp targets, and sufficient selections are performed such that at least 16 billion unique zinc figure-DNA substrate interactions may be present and analyzed for expression of a marker. In an embodiment 384 selections are made.

The disclosure includes zinc figures that comprise at least a zinc finger comprising an amino acid F3 [domain 2] sequence identified by the described method. The disclosure also includes a contiguous polypeptide comprising a set of zinc fingers, one of which comprises an amino acid sequence of an F3 [domain 2] identified by a described method, and a second of which comprises an F2 [domain 1] that was also present in a described assay. The described contiguous polypeptide can bind with specificity to a 3 bp segment of a DNA substrate that was also present in the assay. The contiguous polypeptide can also comprise an F1 [domain 0] that was present in an assay. This contiguous polypeptide can also bind with specificity to the 3 bp segment of the DNA substrate.

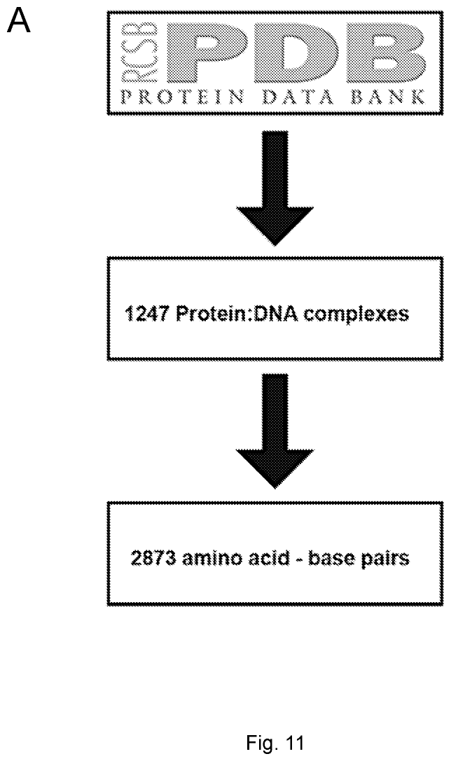

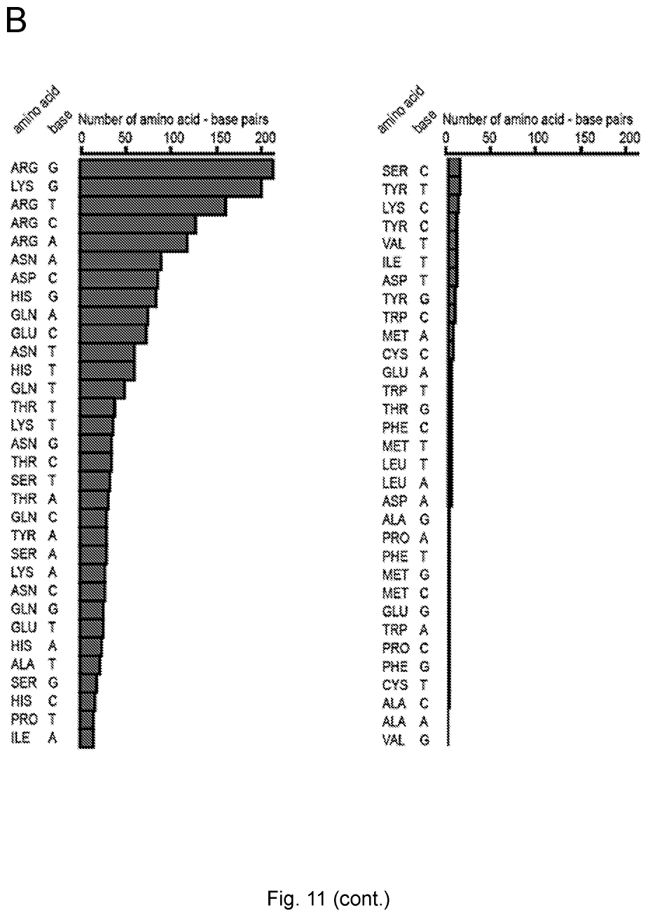

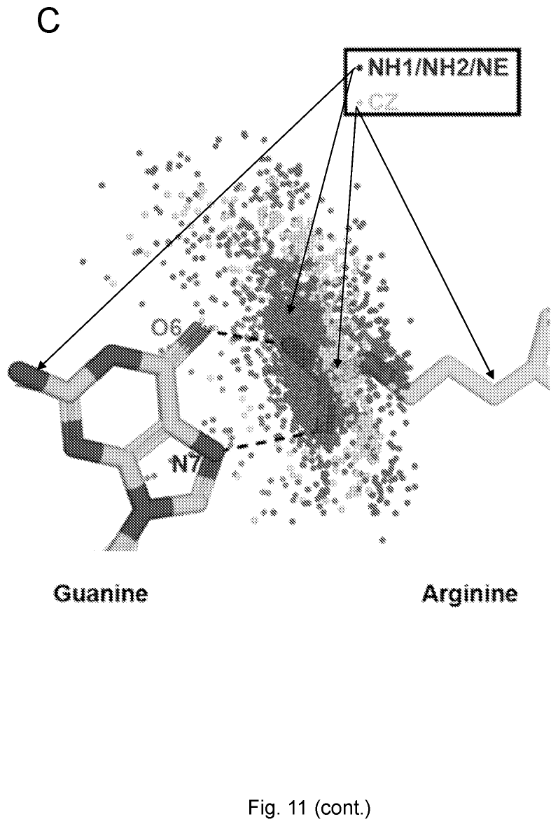

To expand on the foregoing description of distinctions of the present approach a prior methods, two general approaches have been previously used: one focused on engineering one finger at a time and a second approach focused on the interface between adjacent ZFs of an array (FIG. 11). The first approach allows for a comprehensive screen of all amino acid combinations at the six critical positions of the ZF alpha helix (FIG. 7B) but, as the influence of adjacent ZFs is well documented, this approach is limited by the singular adjacent-finger context employed that has primarily been the adjacent influence of an arginine-guanine interaction. As a result, only ZF strategies enabled by this initial selection environment are available in subsequent rounds of selection or as the foundation of a ZF model. The second approach captures the compatibility at the interface between ZFs but is limited by scale (FIG. 7C). Previous approaches that use this approach necessitate a reduced complexity, using an incomplete randomization scheme at only a handful of helical positions. Therefore, the present disclosure provides a combined approach using multiple comprehensive libraries that each fully randomize a single finger's helix, but place that helix in a set of unique interface environments, that produce diverse portfolios of general and interface-specific ZF solutions from which compatible ZF pairs could be generated for a wide range of DNA targets (FIG. 8).

In more detail, the majority of previous ZF engineering efforts come from an era that predates next generation sequencing and therefore methods for the comparison and visualization of ZF data on this scale have not been previously established. For example, the present disclosure provides 12 comprehensive screens of 10 ZF libraries that each represent a unique interface environment in order to provide the helical diversity required to generate compatible ZF pairs across a wide range of targets. We screened these libraries across each of the 64 possible 3 bp target for functional helical strategies. In total the disclosure includes screening over 49 billion protein-DNA interactions across 768 independent selections. We used Hamming distance and position specific scoring matrix (PSSM) to quantify differences in the ZF populations that were enriched across binding site and library environments. However, the inter-dependencies of adjacent amino acids on a ZF helix to specify a given DNA target can result in a convoluted picture of multiple sequence motifs or “strategies” able to bind each target. To deconvolute this picture requires clustering but with data sets this large, the resulting clusters across 768 selections can be difficult to interpret. Thus, the disclosure provides a visualization method to represent the over 1 million functional helical sequences that were recovered on two-dimensional maps with common coordinates. These coordinates were determined by biochemically derived descriptors. By doing so, the data for each selection can be compared visually across the 2D landscapes that describe the functional ZF strategies for a given target under the adjacent finger influence provided by that library. In addition, the disclosure provides for charting where computationally derived clusters are located in this 2D space to help determine the resolution of the clustering. These comparisons permit visualization of both general and interface-specialized solutions to bind most targets. In addition, these comparisons help to reveal that targets with higher G-content tend to offer more general strategies across all libraries screened. This likely explains why the success of prior ZF screens have been biased towards G-rich targets, as these solutions appear to be compatible across most interface environments.

To test whether the described interface-focused screening approach would provide the complexity required to find compatible ZF solutions across a wide range of targets, we followed initial screens with a series of second round, two-finger selections. From these selections we were able to enrich compatible pairs across all targets tested. We assembled these ZF pairs as extended arrays to create a series of 42 zinc finger nucleases (ZFNs) and find that all constructs produced activity above background. The mean activities across all ZFNs tested was similar to the activities reported for TALEN and Cas9 screens in the same assay. Together these data demonstrate the utility of comprehensive screens and the necessity of sampling multiple interface influences in the primary ZF screens in order to provide the complexity necessary for down-stream compatibility. Additional description of these and related approaches are described in the Examples below. In parallel, we have shown that the data described here has sufficient complexity to train an AI-based model for the simple design of ZFs to be employed as nucleases, activators, and repressors.

With respect to improved ZF analysis by using combinatorial screening, in embodiments, the disclosure provides for a total number of selected helices that range from 128 thousand to over 1 million helices per library screened.

In embodiments, the disclosure provides for screening of at least 4 libraries. In embodiments, the 4 libraries each provides a different overlap environment (e.g., one library for A, one library for C, one library for G, and one library for T). In embodiments, this approach comprises use of all possible 3 bp substrates, e.g., 64 bp substrates. In embodiments, each library comprises 64 million sets of ZFs, and would therefore include analysis of approximately 16 billion ZF/DNA interactions (e.g., 4 libraries with 64 bp (all 3 bp) substrates, with 64 million amino acid combinations would yield 16.384 billion ZF/DNA interactions analyzed). In embodiments, the disclosure includes analysis of 4-12 libraries. As another non-limiting example, for 12 libraries analyzed using the described approach, the disclosure provides for analysis of approximately 49 billion ZF/DNA interactions (e.g., 12 libraries with 64 bp substrates, with 64 million amino acid combinations would yield 49.152 billion interactions analyzed. In a non-limiting embodiments, the libraries may include five domain 1 interactions that bind A at the interface, five that bind C at the interface, and 3 that bind G. Libraries that bind T at the interface may also be included. Representative and non-limiting examples are provided below. For instance, as described further below in the Examples, the disclosure demonstrates screening libraries across each of 64 possible 3 bp targets for functional helical strategies, thereby providing analysis of over 49 billion protein-DNA interactions across 768 independent selections.

The following Examples are intended to illustrate but not limit the disclosure.

### Example 1

Adjacent Finger Influences have Global Impacts on Specificity

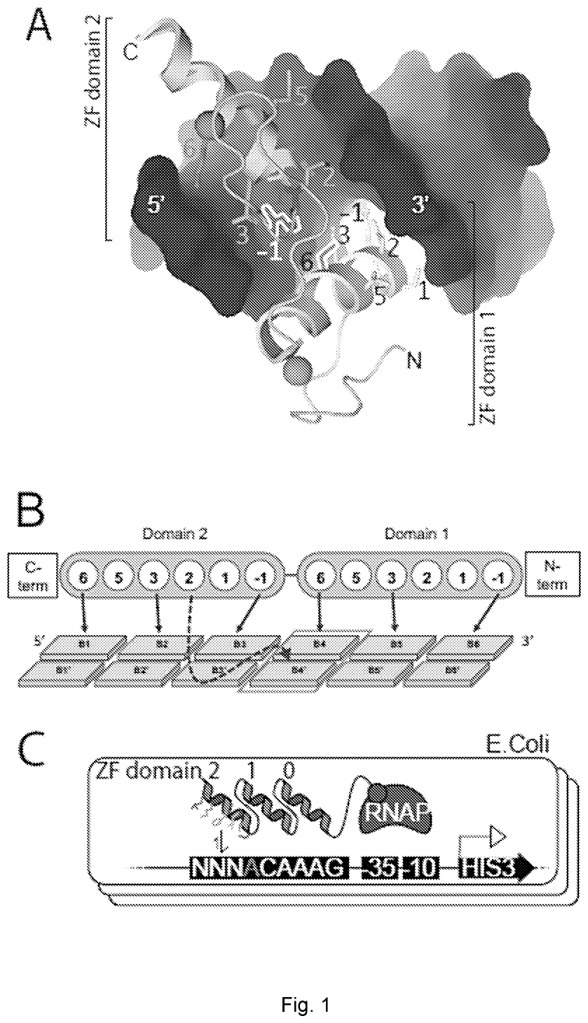

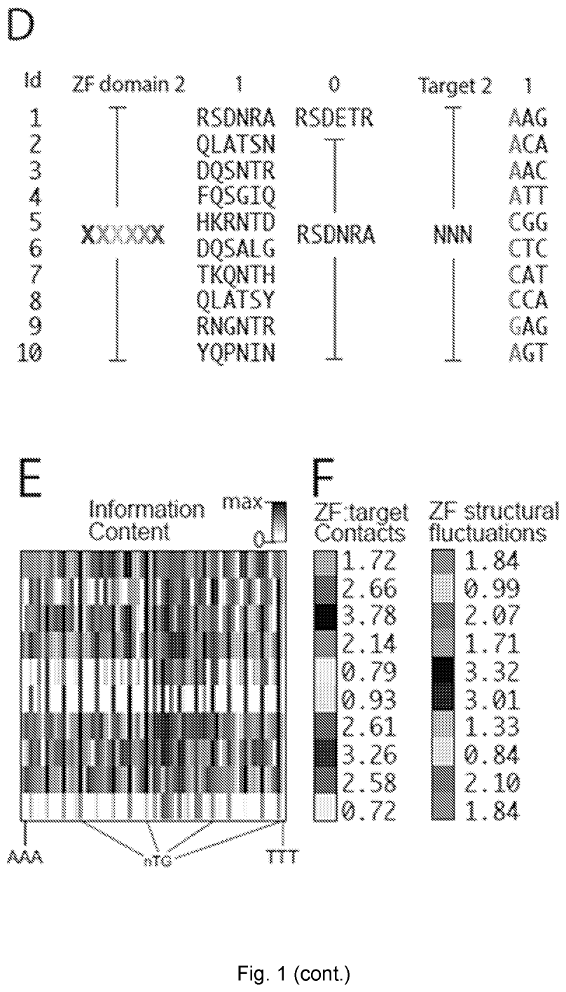

The first structures of ZF arrays bound to DNA demonstrate the influence that adjacent fingers have on one another (FIG. 1A), but a comprehensive screen of these influences has previously been lacking. We previously used an exhaustive screen of the ZF helix, sampling all combinations of the six helical residues that have base-specifying potential, to produce the most accurate model of ZF specificity at the time Persikov, A (2015) Nucleic acids research, 43(3), 1965-1984. However, since all selections were done in a common adjacent-finger context, it failed to capture these important between finger influences. To comprehensively investigate the adjacent finger influence we scaled the approach to screen multiple libraries, each presenting a different amino acid-nucleotide interaction at the interface immediately adjacent to where library members bind (FIG. 1 and FIG. 8). As the majority of published ZF screens have been done in G-rich binding contexts, including selection of ZFs immediately adjacent to fixed arginine-guanine contacts, we expanded the library designs to include those that would specify adenine or cytosine at this “overlap” in addition to the common arginine-guanine contact (see FIG. 1B, overlap base pair is boxed). We screened each of these libraries using a bacterial one-hybrid assay that positions the library finger, the 3^{rd} in an array, immediately adjacent to two ZFs that have known, fixed interactions with their target sequences (FIG. 10). In this way the library finger is positioned directly over the 3 bp target that is varied in each target selection. Each library is screened in 64 independent selections to find which amino acid combinations are able to bind each 3 bp target with sufficient energy to activate the reporter gene. Each library differs only by the fixed interaction between the middle finger and the DNA as this presents a novel interface environment between the middle and library fingers (FIG. 1D and FIG. 8). We designed these libraries to provide novel interface environments that bind A and C at the overlap and present a range of biochemically differentiated side chains including hydrophobic, aromatic, basic, acidic, and polar amino acids (FIG. 8). Each of the fixed interactions presented were taken from previously characterized ZFs tested in the same system. Together, we have captured the strategies that are possible to bind every 3 bp target across 12 unique adjacent finger environments.

We found global and target-specific differences across these libraries. The total number of selected helices ranged from 128 thousand to over 1 million helices per library screened (FIG. 8). To gauge the relative success of each selection we calculated PSSMs for each of the 768 selections and find that low and high Information Content trended by library (FIG. 1E). In addition, molecular dynamic simulations suggest more contacts between the fixed, middle finger and the DNA correlates with global library success (FIG. 1F). Interestingly, selections for most G-rich targets, especially nnG targets, successfully enriched for helices across all libraries (FIG. 1E). Together these results suggest that the success of a selection is at least partly influenced by a baseline affinity provided by the fixed adjacent fingers and, for weaker library contexts, the low baseline affinity can be overcome with a strong guanine contact provided by the selected library finger. However, and without intending to be constrained by any particular theory, while one explanation for low information content is that the selection failed to enrich for functional ZFs, an alternative explanation is the potential presence of multiple, disparate strategies to bind the same target. To clarify, we used MUSI to define ZF clusters for all library selections and used the presence of at least one cluster that demonstrates low entropy as our definition of success. From this analysis we find the same trends and that the success of a library to enrich helices, both globally and at the cluster level, appeared to be library specific. In addition, the success of a library screen does not appear to be dependent on the base specified at the overlap position. At least one library that bound either A, C, or G at the overlap successfully enriched helices in over 95% of the selections (libraries 1—A overlap, 7—C overlap, and 9—G overlap). Conversely, libraries 6 (C overlap) and 10 (A overlap) were the least successful libraries (FIG. 8). These results indicate ZF function is significantly impacted by the adjacent finger interaction but global success is not determined by the overlap base specified.

### Example 2

### The Overlap Base and Specificity Strategies

Zinc finger strategies were enriched in over 85% of the 3 bp target selections for 9 of the library screens. In fact, for every 3 bp target, ZF strategies were enriched in at least 8 different library screens. However, it is not immediately clear if the ZFs enriched to bind a given target are the same or different across libraries or how the overlap base influences the enriched strategies. Therefore, to make a quantitative comparison we measured the relatedness of each selection using hamming distance (FIG. 9). From these comparisons we are able to observe that while there are trends that suggest ZFs enriched in libraries that share a common overlap base are more similar, these trends are not absolute (FIG. 10). Further, there are significant differences between libraries that share a common overlap base. Interestingly, two libraries were able to bind 2 different bases at the overlap position (library 1: A or C and library 3: A or G). We carried out two complete screens with both of these libraries, each with the different tolerated base at the overlap position. For library 1 we found that ZFs enriched across target selections were very similar, regardless of whether the overlap base was an A or a C (FIG. 10B). However, for library 3 we found the selections with A or G at the overlap were quite different. As this difference is observed in a library that places an arginine-adenine or arginine-guanine contact at the interface, we compared all libraries screened to the one other library in our collection that presented an arginine-guanine contact at the interface, library 9. The two arginine-guanine overlap libraries are extremely similar and significantly different from all other libraries screened, including the other screen of library 3 that placed an adenine at the overlap (FIG. 10C). These results suggest that globally, the ZF strategies enriched in all of the A and C overlap libraries are significantly different from the arginine-guanine overlap that has been used in most prior ZF library screens.

### Example 3

### Visualizing Selected Helices in 2-Dimensional Space

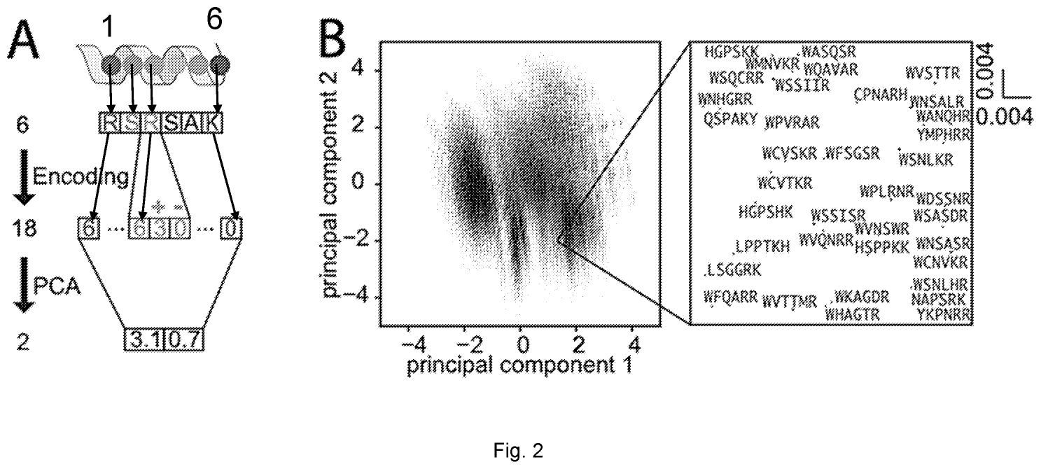

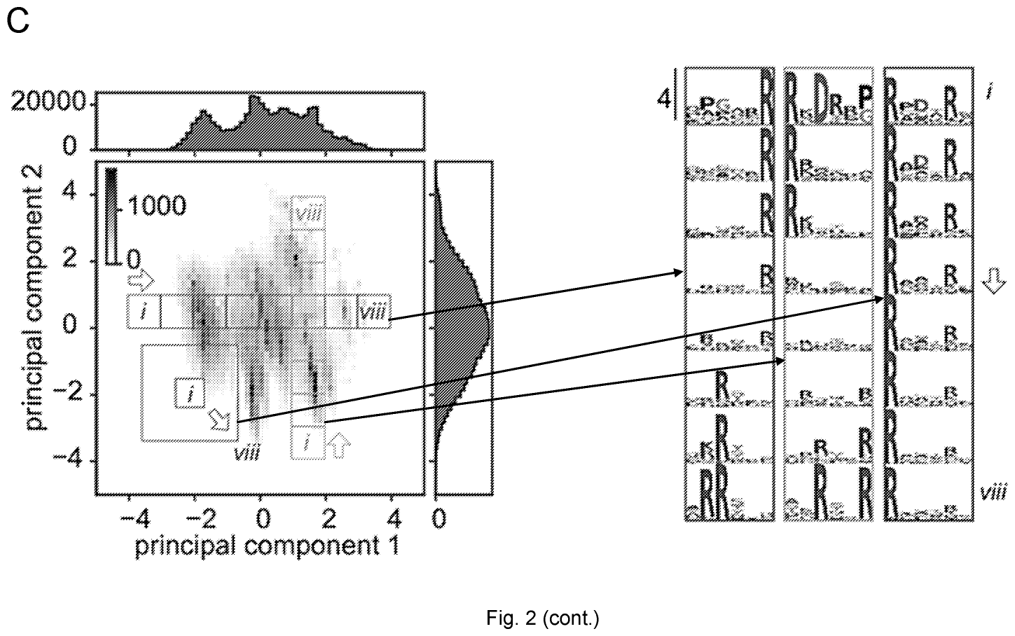

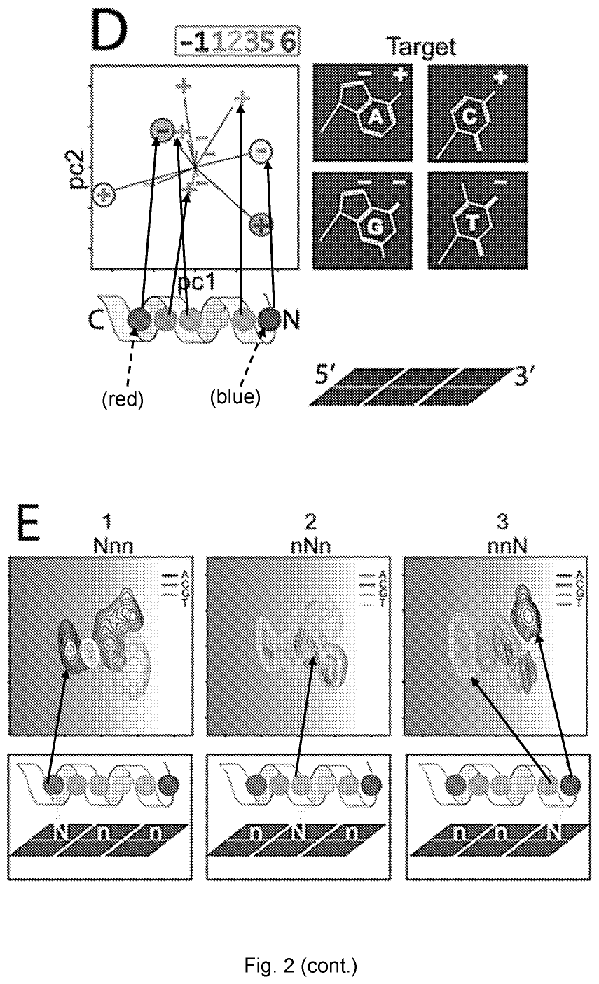

To visualize common and interface-specific strategies enriched to bind any 3 bp target we first used the MUSI generated sequence clusters across all selections. However, this resulted in a cumbersome comparison of thousands of clusters across the 768 selections. Therefore, to more easily visualize and compare helical families across selections we developed a method to distribute selected helices on a common 2D space. In particular, this method is tailored to classify helices by their potential biochemical interaction with DNA bases. First, we encoded each of the six amino acid sequences into numerical descriptors based on physics features. As helical side chains interact with bases in the major groove through combinations of hydrogen bonds and hydrophobic contacts, we used an encoding system where each residue is encoded by three integers: (i) the number of hydrogen bond donors, (ii) the number of hydrogen bond acceptors and (iii) the side chain length from the Ca to the most distal atom (FIG. 2A). We tested this encoding system with a ten-fold cross validation predicting the correct binding site among all possible 64 triplets for 47% of helices. The addition of a fourth feature accounting for hydrophobicity did not improve the prediction, likely reflecting that hydrophobicity is correlates with the absence of hydrogen bond donors and acceptors. For visualization, we reduced the resulting N×18 matrix, where each of the N helices is encoded with 18 normalized descriptors, to N×2 based on a Principal Component Analyses so that we could plot this information in 2D space. The first 6 principal components explain 14%, 11%, 10, 9%, 8% and 6% of the whole variance. This range may be explained by the roughness of the sequence space and the low Information Content at the helical positions 1 and 5 that have a lessor impact on specificity relative to positions −1, 2, 3, and 6.

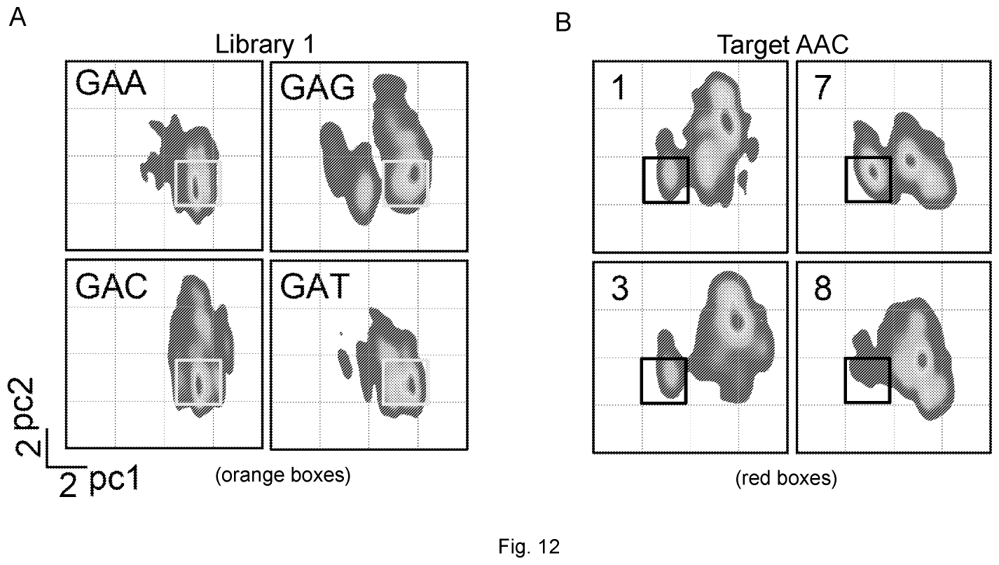

We created a PCA map using these descriptors that includes over 1 million helices recovered in the 768 selections. This map allowed us to track the coordinates of any selected helix, or related helix, and their relative positions on the 2D coordinates of the map. As positions 1 and 5 of the helix have less of an influence on specificity, variability at these positions allow for up to 400 version of any helix with common “core” residues suggesting that the map reflects multiple versions of any core helix (a core helix is defined as any helix with the same residues at positions −1, 2, 3, and 6). From this map we find that similar helices are found with similar coordinates (FIG. 2B). Further, restricting the search to various regions of coordinates shows coherent sequence logos and the important role of arginine at multiple helical positions for specificity and likely stabilizing affinity (FIG. 2C). The enrichment of arginine was not unexpected based on results described above as analyses of a thousand structures from protein:DNA complexes confirmed that arginine is the most prevalent residue binding DNA bases (FIG. 11). The projection of the features used as descriptors along pc1 and pc2 shows the critical contribution of the position −1 and 6 (FIG. 2D) as donors and acceptors are found on opposite sides of these axes for these helical positions. Furthermore, helices scatter according to their specificity (FIG. 2E) demonstrating similar helical features are associated with similar target preferences. For instance, helices that recognize a guanine at positions 1 and 3 of the target DNA, a base with two hydrogen acceptors scatter separately from helices binding a cytosine at these positions, a base with one hydrogen donor. Both groups of helices segregate along the direction of the hydrogen bond features of the residue binding that base. Conversely, the helical scatter is more complicated for specificity at the middle base of the ZF target. These results suggest that the display of the helices recovered in any individual selection on these PCA coordinates will allow a visual comparison of related helices and the organization of these helices is related to their specificity due to the encoding system that relies on hydrogen bond potential. These comparisons can be useful to identify helical families that provide refined specificity for similar targets (FIG. 12) and the comparison of helical families that bind the same target under the influence of different library contexts (FIG. 12B).

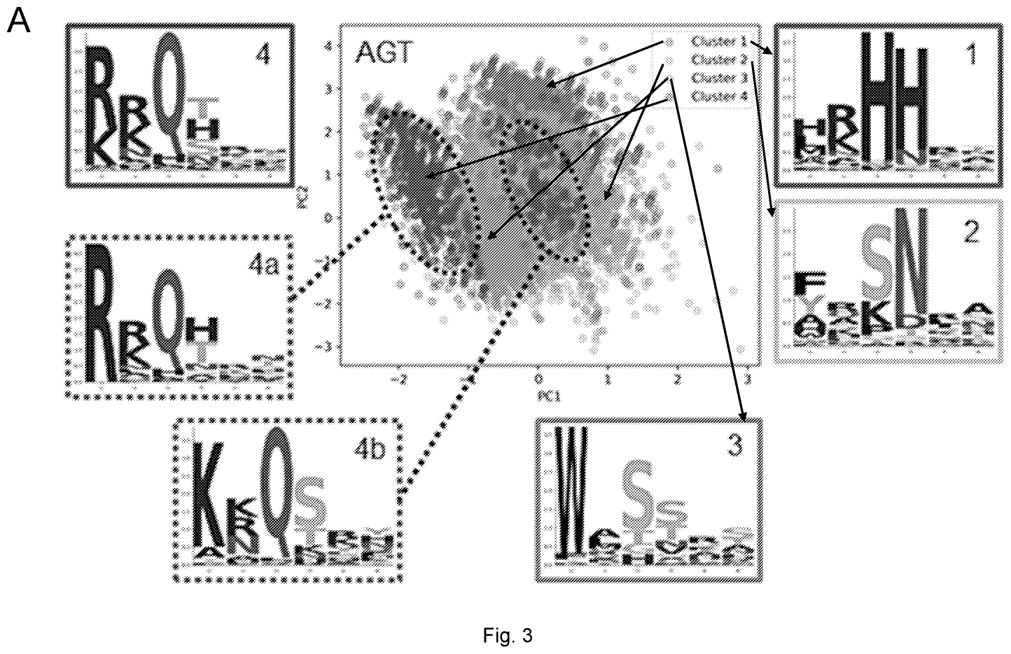

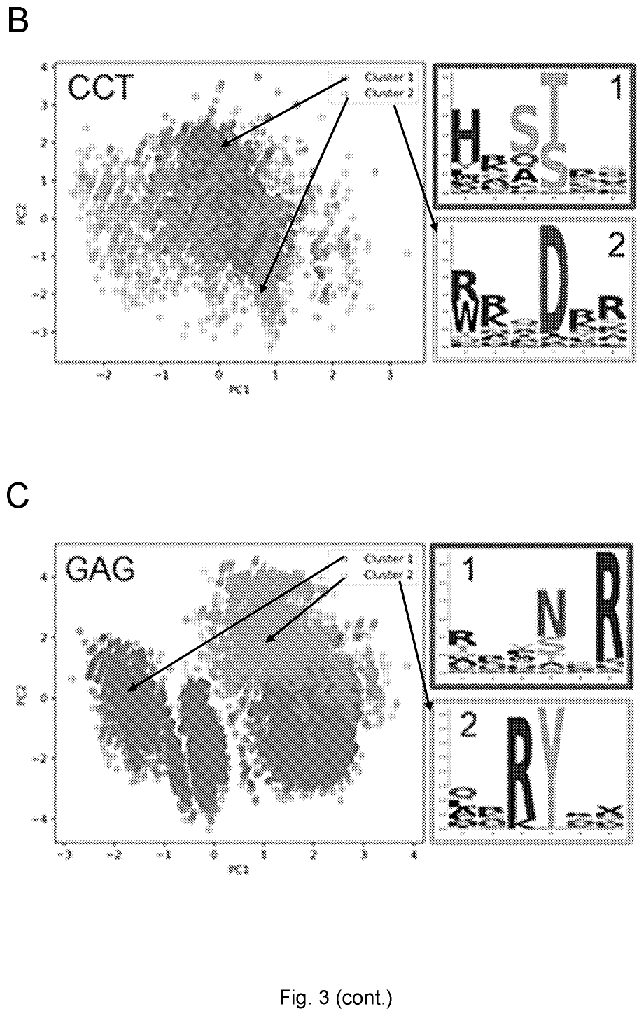

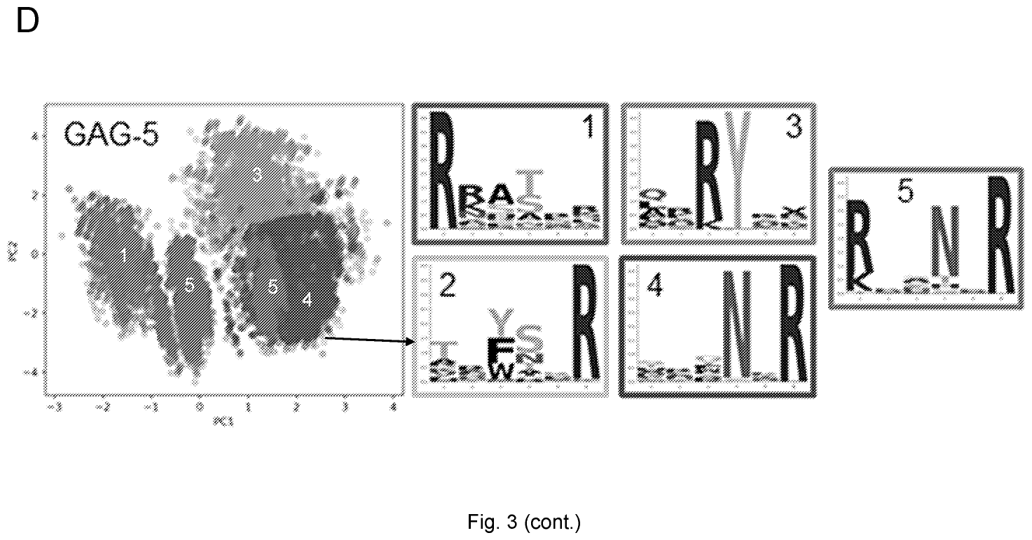

An object of clustering and visualization of the enriched helices on the PCA maps is to identify helical sequences where amino acids are dependent on one another and thus represent unique binding strategies. This is believed to be necessary as all of the positions on the ZF helix have the potential to influence one another and therefore, the enriched residues should not be thought of as independent but rather a network of interactions as the entire helix interacts with the target. To confirm that the PCA maps separate related helices we plotted the location of the ZFs included in the MUSI-determined clusters on each selection-specific PCA map. In general, when MUSI determines that there are 3 or less clusters, the PCA maps separate most clusters into localized regions of the map. For selections with 4 or more cluster, a subset of the clusters will tend to overlap (FIG. 3A). However, there are exceptions to these trends. In some case, even two clusters will have significant overlap (FIG. 3B). Conversely, there are several cases where a MUSI-defined cluster is separated in groups on the PCA map suggesting different families within the cluster (FIG. 3C). This is far more common for G-rich binding sequences that are dominated by a single arginine-guanine contact. A closer investigation indicates that some, but not all, of these families do represent different subfamilies of helices where alternative amino acids have coevolved. Forcing MUSI to include additional clusters can separate some of these groups (FIG. 3D). In summary, plotting the recovered helices on a common 2D landscape as described herein provides a convenient, visual comparison that can provide meaningful insight as far as the differences and similarity of evolved helical families while simultaneously indicating when more computationally defined clusters are desirable to reflect the recovered data.

### Example 4

### Adjacent Fingers Influence General and Specialized Strategies for DNA Targets

Globally, we compared the target selections in three ways: the ability to enrich functional helices, the quantitative comparison of hamming distance, and the qualitative comparison of PCA maps that provide a 2-dimensional representation of the specificity landscape. From these maps we can easily visualize populations that are differentially enriched across similar targets within the same library or within the same target across multiple libraries (FIG. 4A and FIG. 12). Therefore, from these comparisons we can visualize general and specialized ZF strategies for any given target while quantifying the relatedness of all strategies employed by one library versus another. Depending on the target sequence, we find a wide range of relatedness across these selections (FIG. 9). Interestingly, populations that bind A/C/T's exclusively tend to be less related, (FIG. 4B, left) suggesting that for these targets adjacent finger compatibility plays a larger role. Conversely, populations that bind G-rich targets tending to be more related, this is especially true for nnG targets (FIG. 4B, right). These trends can be visualized through the enrichment of common populations seen in the PCA plots as well as the hamming distance comparisons (FIG. 4B, bottom). In fact, 14 of the possible 16 nnG targets are found in the 18 target selections with the lowest normalized hamming distance while no G's are found in the 9 binding sites with the highest scores (FIG. 4C). These results demonstrate that G-rich binding is less influenced by the adjacent finger which may help to explain why the success of prior engineering has been biased towards the selection of these types of ZFs.

### Example 5

### Zinc Finger Incompatibility is Demonstrated Across Selections

Hamming distance and PCA plots reveal trends in general populations that are enabled across most library contexts as well as more specialized populations that are specific to subsets of libraries. These specialized populations even appear within the class of highly similar nnG selections where most, but not all, solutions appear to be general (FIG. 5A). We focused on two examples, GTG and CCG, to demonstrate the contrast and suggest a mechanism for differences observed. For the GTG target, 3 primary groups of helices appear to be enabled across all library selections at various frequencies (FIG. 5A). The G specified at the third base (GTG) is typically recognized by an arginine at position −1 in these groups. As this interaction brings the positively charged arginine close to the negatively charged DNA, and it presents the potential for two hydrogen bonds between the arginine and the guanine in the major groove, this contact provides a stable and high affinity interaction documented throughout the ZF literature. However, in some, but not all, CCG selections an additional cluster appears consisting of QxRYxx helices that do not use an arginine at position −1 to specify the G at the 3^{rd} position of the binding site (FIG. 5A, bottom). From the PCA plots it is immediately visible that this group of helices does not occur in the library screens where an arginine is expressed at position 6 of the adjacent finger. To understand this difference, we performed molecular dynamic simulations for the helix QSRYTT (SEQ ID NO:1) in the context of library 2 (N6) and library 3 (R6). The simulations suggest that when position 6 of the adjacent finger is anything other than an arginine, the arginine at position 2 of ZF domain 2 (QSRYTT) (SEQ ID NO:2) is able to interaction with the 3^{rd} base of the target CCG, with a suggested 2 hydrogen bonds in the majority of simulations (FIG. 5B, top and 5C). Conversely, in the context of a library where an arginine is at position 6 of the adjacent finger (ZF domain 1), there is a competition for binding the G at the 3^{rd} position of the binding site, CCG. This results in a much lower frequency with which position 2 of domain 2 is able to make the arginine-guanine contact (FIG. 4B, bottom). In addition, the frequency that the arginine at position 6 of domain 1 is engaged with the B3 position of the target (FIG. 5E) will decrease the availability of that residue to interact with the canonical B4 interaction of domain 1. Therefore, this conflict may decrease the affinity of both fingers simultaneously and explain the absence of this strategy from all R6 library selections. We also observe that this trend is not exclusive to CCG but is seen in all 4 of the nCG target selections as the simulations suggest the tyrosine of the QxRYxx motif interacts with the cytosine at the B2 position (FIG. 5D). Finally, while contacts have been noted between position 2 with the B3′ and the B4′ bases of the model (FIG. 5E), the suggested contact with the B3 base represents a novel interaction. This is also true for the suggested, conflicting interaction between position 6 of domain 1 with B3.

### Example 6

### Compatible ZF Pairs are Enriched Across a Wide Range of Targets

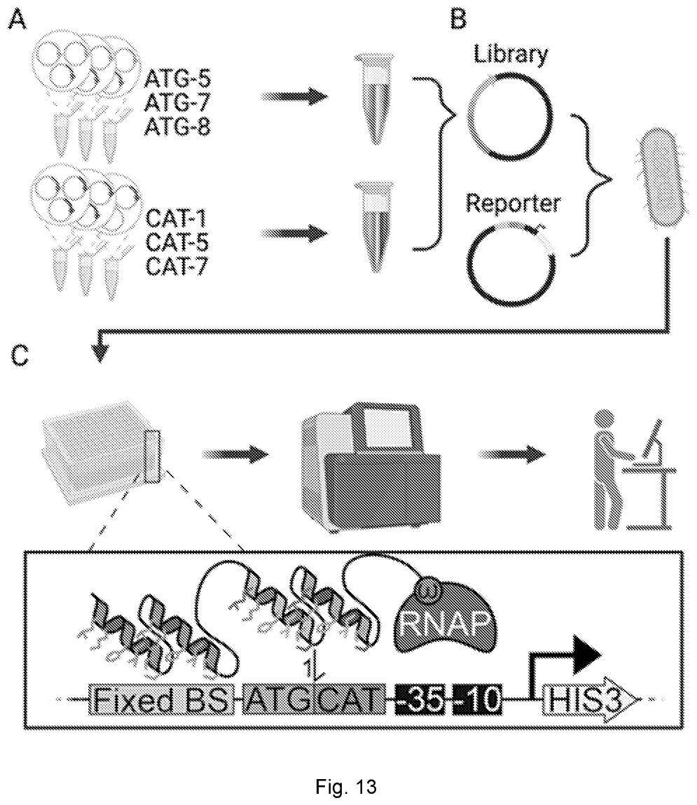

The described library screens demonstrate the profound influence that adjacent fingers have on one another despite their restriction to a relatively small subset of adjacent finger influences. However, we reasoned that the wide range of biochemical environments that these libraries represent, and the diversity of helices enriched across these selections, would provide the complexity necessary to uncover compatible helices across a wide range of targets. Therefore, to consider compatibility on a more comprehensive scale we created pools of ZFs for each 3 bp target that included helices from all successful library screens. From these pools we generated 2-finger libraries compatible with 178 6 bp targets (FIG. 13). These 2-finger libraries represent helices that have been pre-selected to bind each 3 bp sub-target for the 2-finger protein. We used these libraries then to screen for compatible ZF pairs for all 178 targets with a modified version of our B1H assay that includes enrichment in selective liquid media. The enriched pairs were then sequences.

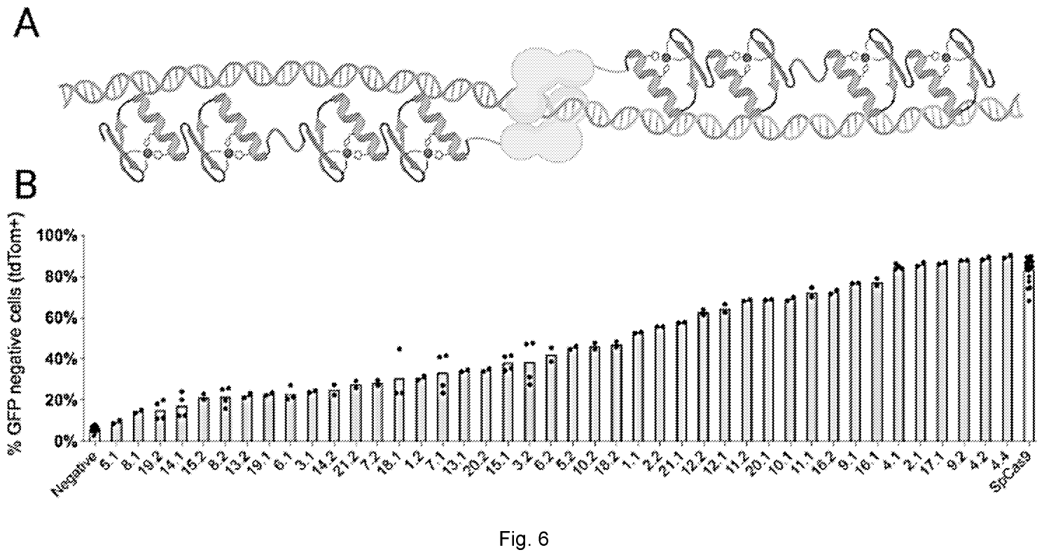

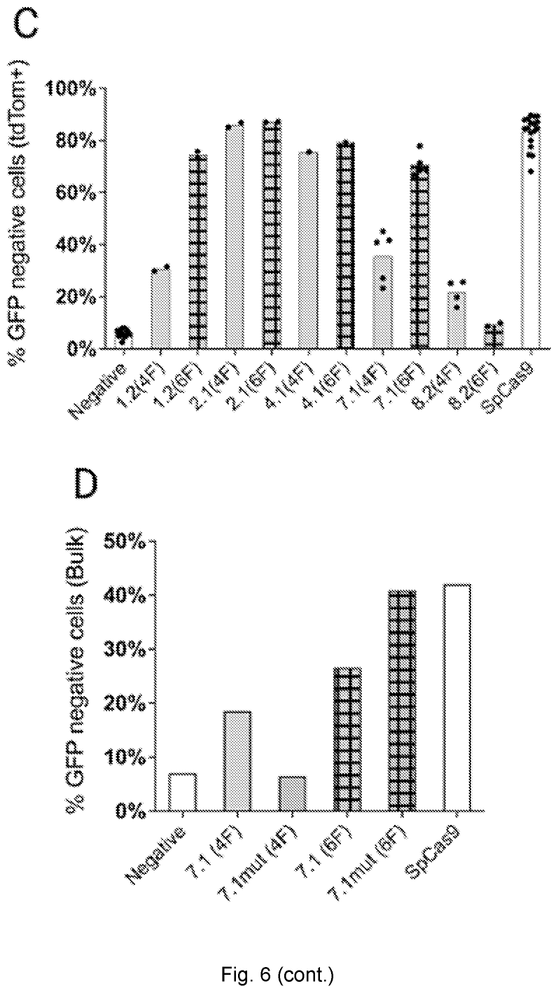

To demonstrate that the compatible pairs are functional outside of the bacterial selection context, we use the pairs to create a series of 42 zinc finger nuclease (ZFNs) that target the coding sequence of eGFP. In order to maintain the independence of binding for one ZF pair versus another, we used a linker to separate each pair that skips a base between their targets. In this way we have reduced the interface compatibility issue for ZFs to a 2-fingered problem across the entire array (FIG. 6A). As a result, each monomer expressed 2 sets of ZF pairs fused the fok1 nuclease domain, providing 12 bases of specificity per monomer and 24 bases total. We screened these ZFNs for their ability to disrupt the GFP coding sequence in a human U20S cell line that has a copy of an eGFP-PEST construct integrated into the genome. As indels in the coding sequence can lead to frameshifts and loss of fluorescence, this assay has been used to approximate nuclease function for both TALENs and spCas9. When we assayed these 42 ZFNs that target sequences in the GFP coding sequence (FIG. 6B), the panel of ZFNs resulted in loss of fluorescence that ranged from 13% to 86%, with a mean of 48% for all constructs. In addition, for ZFNs that produced low activity, by extending each array to include 6 ZFs per monomer increased the ZFN activity in 2 or 3 examples while extending highly active ZFNs had no influence (FIG. 6C). In the one case where activity decreased, the ZFN was one of the weakest performing constructs, likely due to low affinity or specificity. In either case, the additional ZFs may have decreased on-target activity due to increased off-target binding by the additional ZFs. One strategy to address off-target binding has been to decrease the non-specific affinity provided by residues that make phosphate contacts such as the arginine at position −5 of the ZF (numbered relative to the start of the helix). To test the impact on non-specific affinity we substituted the arginines at the −5 position of a set of ZFs for a ZFN with low activity. For the 8-fingered version of this ZFN (4F per monomer), this reduction in nonspecific affinity had a negative impact on activity suggesting that the baseline affinity was too low for this construct (FIG. 6D). However, the 12-fingered version of this same ZFN showed an increase in activity by replacing these phosphate-contacting residues, presumably by reducing off-target activity. These results demonstrate that even for weak ZFNs generated by this method their activity can be improved by extending the array and/or reducing the non-specific affinity. This work represents the most consistent production of ZFNs with high activity to date demonstrating how ZF compatibility, and therefore sampling different primary selection environments, is a generalizable and highly functional method to address ZF engineering across targets.

### Discussion of Examples

It will be recognized from the foregoing description that the present disclosure provides an alternative approach to maintain maximum diversity in the randomized ZF but systematically changing the environment presented by the adjacent finger. Among other aspects, the disclosure provides contexts that position the library adjacent to fixed interactions with A's and C's at the interface. This takes into account how these interactions would provide critically differentiated environments, compared to the commonly explored arginine-guanine environment, to generate novel binding strategies that would provide compatible ZF options for a wide-range of target sequences. Indeed, the described results demonstrate that this approach was successful as ZF populations enriched using libraries with A and C overlaps were all significantly different from libraries that presented arginine-guanine contacts at the interface (FIG. 10C). In addition, libraries that specify the same overlap base can still demonstrate substantial differences in activity and specificity across targets suggesting that both the base specified and the environment provided by the side chain (basic, acidic, polar, or hydrophobic) have profound influences on ZF function, compatibility, and specificity. Further, G-rich targets were the least influenced by the adjacent finger contexts. These results suggest a plausible explanation for why prior engineering efforts have been biased towards G-rich interactions as these ZFs were evolved in one context, but when moved out of that context, the G-rich binding ZFs are influenced the least by context and are therefore the most likely to be functional in a multitude of new environments. Conversely, the present data suggests ZFs that bind A/C/T sequences are very much dependent on their adjacent finger context and would have been more likely to fail in a new environment. Together these results demonstrate the presently described value of considering a wide range of interface environments to understand ZF compatibility.

A benefit of the presently described approach was confirmed by selecting a series compatible 2-fingered modules for 178 6 bp targets from libraries generated from these primary, interface focused selections. By choosing the most enriched pairs from each of these selections we were able to produce a set of 42 ZFNs with a range of activity similar to what would be expected from a series TALENs or a Cas9-based screen of guide RNA's. Further, we demonstrate that weak ZFNs can be improved by extending the array or reducing non-specific affinity that limits off-target binding. Since the primary selections were mostly focused on A and C at the overlap, we maintained this preference in the 6 bp targets specified for the ZFNs in our screen. We also confirmed that the G-content for the most active targets is no different than random, suggesting that the fingers are not limited by G-binding as previous efforts have been. The use of a linker to skip bases between these modules allows each module to function independently. Therefore, a complete set of 4096, independent 2-fingered modules could be generated based on this approach and applied to any target sequence. In parallel, we have shown that the data produced in this disclosure offered enough complexity to generate an AI-based model of ZF specificity that enables their simple design for application as nucleases, activators and repressors.

The following materials and methods were used to produce the data described herein and depicted in the figures.

### Library Builds

Primary zinc finger libraries: All primary ZF libraries were built as previous described and detailed below. To provide templates for PCR, gBlocks were ordered from IDT that coded for the finger 0 and finger 1 domains of each library (FIG. 8, and see FIG. 1c for numbering of domains). Differences that distinguish each library from one another is that they each place a different environment at the interface between domain 1 and the library domain 2. These libraries include five domain 1 interactions that bind A at the interface, five that bind C at the interface, and 3 that bind G. These libraries use side chains at the interface with a range of biochemical properties to interact with the overlap base (basic, acidic, polar, aromatic, and hydrophobic interactions). Together, the biochemical property of the side chain at position 6 of domain 1 and the base it specifies at the overlap position represent the unique interface environment offered by each library. Next, an oligonucleotide was design with degeneracy at the codon positions corresponding to the six critical residue positions of the ZF domain 2 alpha helix. This oligo was used for all library builds, only the template gBlock, and therefore the finger 0 and 1 domains, are changed. PCR was used to generate the library insert, amplifying from the library-specific gBlock template with the library oligonucleotide paired with a downstream oligonucleotide used to capture the full 3-finger insert. For each library, PCR reactions were run in 96-well plate format and pooled. The PCR products were digested with Kpn1 and XbaI and ligated into 15 μg of digested B1H expression vector. Ligations were run over night at 16° C., ethanol precipitated, and resuspended in 15 μl of 10 mM Tris-CI, pH 8.5. The ligation was electroporated into 15 aliquotes of electrocompetent US0 cells and recovered in 1 L of SOC. One-hour post electroporation, 200 μl of the culture was titered in 10-fold serial dilution on Carbenicillin plates to determine library size. To select for transformants, carbenicillin was then added to the culture at this point and grown to mid-log. The library DNA was then recovered by Qiagen maxiprep. Library sizes ranged from 1-3×10⁹. This approach has been shown to consistently produce libraries with diversities that approximate random.

2-finger libraries: Second round selections were used to select compatible pairs from pre-selected ZF pools generated in the primary ZF library selections. We pooled recovered plasmid DNA from our primary single-finger screens on a binding site basis, resulting in a pool of diverse helices (termed “round 2 pools”) with broad compatibility for each of the 64 different binding sites. To ensure these were enriched for functional helices and not background, a simple cutoff was devised to omit unsuccessful selections. Based on the data filtering metrics described, single-finger pools were omitted if less than 20% of the reads passed these filters as those selections would have added a disproportionate amount of non-functional ZFs to our template pools. A table depicting the pooling strategy is shown in FIG. 12. This set of 64 round 2 pools was used as a PCR template to create either ‘domain 1’ or ‘domain 2’ amplicons using Expand™ High Fidelity PCR system (Roche) and 15 cycles to reduce bias. ‘domain 1’ and ‘domain 2’ reactions were gel-purified from a 2% agarose gel, quantified by nanodrop, and stored at −20 C. In order to create a 2-finger library insert, we performed overlapping PCR to stitch appropriate ‘domain 1’ and ‘domain 2’ pools together. Briefly, purified single-finger amplicons were combined equimolar as the template for overlap PCR with Phusion® High Fidelity DNA Polymerase (NEB) (25 cycles), PCR-purified, digested with KpnI and NotI, gel-purified, and quantified by Nanodrop (ThermoFisher Scientific). The digested 2-finger library inserts were ligated into our 2-finger library vector (see FIG. 12). Ligations were performed overnight at 16 C using 300 ng of digested backbone and a 5:1 molar excess insert:backbone. Ligations were ethanol precipitated and resuspended in 5 uL EB (Qiagen). 100 ng of the ligation was electroporated into USO-ω cells, recovered in SOC for 1 hr, titered on 2×YT agar plates containing 2% glucose and 100 ug/mL carbenicillin, and stored at 4 C overnight. Based on cell counts the following day, 5×10⁶ cells were plated on 15 cm rich media agar plates (2×YT, 2% glucose, 100 ug/mL carbenicillin), grown at 30 C for 12-14 hours, harvested by scraping, and finally miniprepped to obtain final round 2 libraries.

### Zinc Finger Selections

Primary ZF Libraries: Libraries were built in a vector that will express the ZFs as a fusion to the omega subunit of the bacterial polymerase using a strong promoter. In the B1H system omega is simply acting as an activation domain. The binding site reporter vectors were built by placing the binding site of interest 10 bp upstream of the −35 box of the promoter that drives HIS3 and GFP expression in the previously described GHUC vector. For example, for the library 2 TAC selection, the binding site 5′ TAC-ACA-AAG 3′ was built into the GHUC vector 10 bp upstream of the promoter where the library domain will bind TAC and domains 1 and 0 of library 2 will bind ACA and AAG, respectively (FIG. 1c). For each selection, the ΔrpoZ selection strain was transformed with the ZF library and the appropriate reporter plasmid by electroporation. The cells were expanded in 10 ml SOC for 1 h at 37 C with rotation, recovered and resuspended in minimal media supplemented with histidine and grown with rotation for an additional hour at 37 C. Finally, cells were washed in minimal media that lacks histidine, recovered in 1 ml of this media, and 20 μI's plated in serial dilution on rich plates containing Kanamycin and Carbenicillin to quantify double transformants. This plate was grown at 37 C overnight while the remaining 980 μl of transformed cells was stored at 4 C. Once grown, the serial dilutions were counted and a volume containing a minimum of 5×10⁸ cells were taken from the transformants stored at 4 C and plated on selective media. These plates contained 2 mM 3-AT, a competitive inhibitor of HIS3, that helps to removed background activity from the screen. Cells were grown on the selection plates for 36-48 h at 37 C. Colonies were counted, cells were pooled, and DNA harvested. This DNA was used as the template for Illumina sequencing. All selections resulted in hundreds to thousands of surviving colonies.

Compatible 2-finger modules selections: In order to identify compatible 2-finger modules from our round 2 libraries, we first built a matching set of vectors containing the intended DNA target and then leveraged omega-dependent activation of the HIS3 reporter in our bacteria 1-hybrid system. Round 2 libraries were co-transformed with the matching reporter vector in USO-ω cells and recovered and titered as described. Based on cell counts the next day, 1×10⁶ cells were added in triplicate to a 96-well deep-well plate containing a sterile bead for efficient agitation. Selections were performed in 1 mL NM +Ura/−His supplemented with 100 μg/mL carbenicillin, 50 μg/mL Kanamycin, 1 μM IPTG, and 5 mM 3AT. These were grown at 37 C in a plate shaker for 18, 24, or 40 hours and harvested upon reaching visible turbidity (typically OD>0.6). Triplicates were pooled, miniprepped, and deep sequenced on an Illumina NextSeq 500. Helices were rank-ordered by sequencing reads, and 2-finger modules within the top 5 highest counts were chosen for follow-up assembly and testing in the EGFP nuclease assay.

### U20S GFP Disruption Assay

Zinc finger nuclease (ZFN) activity was assessed by measuring disruption of an integrated, constitutively-expressed eGFP reporter in a clonal U2OS cell line previously described (Reyon et al. 2012, Nat. Biotech). Cells were cultured in DMEM supplemented with 10% FBS, 2 mM GlutaMAX™ (Life Technologies), 1% penicillin/streptomycin, 1% MEM non-essential amino acids (Life Technologies), 2 mM sodium pyruvate, and 400 μg/mL G418. 1 μg of each ZFN monomer plasmid DNA and 200 ng ptdTomato-N1 plasmid DNA were transfected in duplicate into 5×10⁵ cells using a Lonza Nucleofector™ 2b Device (Kit V, Program X-001). In each assay 2 μg of the parental empty vector (a modified derivative of the JDS71 vector from addgene) and 200 ng ptdTomato-N1 was used as a negative control, and 2 μg of a dual spCas9-guide expressing vector (modified addgene plasmid #41815) and 200 ng ptdTomato-N1 was used as a positive control in each experiment. Cells were grown in 6-well dishes for 3 days post-transfection, harvested and kept on ice, and analyzed for expression of eGFP and tdTomato on a Sony SH800 cell sorter. In order to restrict analysis to only cells that likely received both ZFN monomer plasmids, populations were first gated on the top 15-25% tdTomato+ cells, and then analyzed for loss of eGFP expression.

### Next Generation Sequencing and Prep

Primary libraries: Following selection from >5×10⁸ library variants, surviving colonies were pooled, miniprepped, and DNA barcoded for sequencing on an Illumina NextSeq® 500. Typically these were performed as a set of 64 3 bp binding sites for a given ‘overlap’ library as follows. 2 uL of pooled plasmid DNA was used as a template for barcoding in a 254 reaction with Taq Polymerase (NEB) with the following cycling parameters: 95 C for 5 min, 20 cycles of [95 C:20 s, 52 C:30 s, 68 C:30 s], 68 C for 10 min, and held at 4 C. 54 each reaction was visualized on a 1% agarose gel to confirm apparent equal amplification. All 64 reactions were pooled in equal volumes. These were run out on a 1% agarose gel, gel purified, and submitted to the NYU Genome Technology Center for sequencing on a NextSeq® 500.

2-finger libraries: Following selection of ˜3×10⁶ 2F library variants, plasmid DNA was extracted from surviving cells and barcoded for deep sequencing on an Illumina NextSeq® 500 as follows. 24 pooled plasmid DNA was used as a template for barcoding in a 25 μL reaction with GoTaq® Green 2× Mastermix (Promega) with the following cycling conditions: 95 C for 5 min, 15 cycles of [95 C:30 s, 68 C:30 s, 72 C:60 s], 72 C for 5 min, and held at 4 C. 10 μL each reaction was visualized on a 1% agarose gel to confirm equal amplification, all reactions were pooled in equal volumes. These were gel-purified from a 1% agarose gel, and submitted to the NYU Genome Technology Center for sequencing on an Illumina NextSeq® 500.

### Sequence Recovery and Filtering

All paired end Illumina reads are demultiplexed and trimmed into 21-mers with in-house Unix scripts based on EMBOSS 6.6.0. Trimmed DNA sequences are translated, and amino acid sequences are considered if they have a least two read counts and are coded by at least two different DNAs. The invariant Leucine at the helix position +4 is excluded.

### Clustering and Filtering Selections

For each selection, helix sequences were clustered using the MUSI software. Each sequence was assigned to the cluster associated with the PWM for which it was assigned the highest responsibility. For each cluster generated, the Shannon entropy value was calculated for each helix residue based on the PWM for that cluster. If a selection lacked a cluster with at least one position with an entropy of two or less, that selection was filtered out for downstream analysis.

### Principal Component Analysis and Mapping

Over 1 million different helix sequences are retrieved from all selections. We further examined all these data with Python3.8 and the libraries Numpy 1.19, Pandas 1.2, sklearn 0.23 and logomaker 0.8. Then, each helix sequence is converted into 18 integers where each amino is coded by a combination of 3 physical features—side chain length from the Ca to the most distal atom in the side chain, number of hydrogen bond donors, the number of hydrogen bond donor. We verified that the encoding system was optimal to predict 3-nucleotide binding sites with a Random Forest algorithm and would not gain with additional features, such as the side chain hydrophobicity. We standardized all features before Principal Components Analyses reduction of the feature space. The first six Principal Components capture 0.135%, 0.108%, 0.101%, 0.093%, 0.083% and 0.064% of the total variance highlighting the roughness of the sequence space and the low information content at some helix position. Helices can be classified by a hierarchical clustering on the first two PCA coordinates. More components can be included to capture a more detailed sequence space.

### Computing Similarity Between Selections by Hamming Distance

To compare the helices from two selections, A and B, pairwise normalized Hamming distances were computed between the two sets of filtered sequences based on the number of identical amino acids. The minimum normalized Hamming distance was then computed from each helix in selection A to each helix in selection B as well as from each helix in selection B to each helix in selection A. The overall distance between the two selections was computed as the mean of these distances.

### Molecular Dynamic Simulations

The PDB file 1AAY was used as template, the DNA was elongated by 2 bp at each end using X3DNA to avoid the melting end effect so that the binding of zinc fingers is not affected. The DNA and protein sequences were mutated using Chimera (cgl.ucsf.edu/chimera/) for each library and test case, the protonated states were determined by WHATIF (swift.cmbi.umcn.nl/whatif/) The prepared structures were then solvated into a TIP3P water box with 15-Å buffer of water extending from the protein/DNA complex in each direction, sodium ions were added to ensure the overall charge neutrality. The FF99 Barcelona forcefield was used for protein/DNA complex and zinc amber forcefield for zinc ions. The particle mesh Ewald method was used for electrostatics calculations. The SHAKE algorithm was used to constrain the hydrogen-containing bond lengths, which allowed a 2-fs time step for MD simulation. The non-bonded cut-off was set to 12.0 Å. The systems were energy minimized using a combination of steepest descent and conjugate gradient methods. Then the systems were thermalized and equilibrated for 3 ns using a multistage protocol. The first step was a 1.5 ns gradual heating from 100K to 300 K, followed by 1.5 ns of density equilibration, both at 1-fs step length. Berendsen thermostat and barostat were used for both temperature and pressure regulation for another 6-ns equilibration at 2-fs step length with gradually reduced positional constraints at 300K. The systems were built with tleap and the simulations were conducted with GPU accelerated Amber18. For each system, three 500-ns trajectories were simulated. The hydrogen bond analysis was performed using BioPython. We considered as a hydrogen bonds any contacts below 3.5 Å between the atoms 06 and N7 in a Guanine and the atoms NH1 and NH2 in an Arginine or ND2 and OD1 for an Asparagine. Bifurcated hydrogen bonds between a guanine and an arginine are identified when two pairs 06-NH1/2 and N7-NH1/2 are found, allowing the tautomeric bifurcated hydrogen bond.

While the disclosure has been particularly shown and described with reference to specific embodiments, it should be understood by those having skill in the art that various changes in form and detail may be made therein without departing from the spirit and scope of the present disclosure as disclosed herein.

1. A method of determining amino acid sequences from a plurality of zinc fingers that bind to specific DNA substrates in a DNA sequence dependent manner, wherein the binding of at least some of the zinc fingers in the plurality is determined by expression of a selectable marker and optionally a detectable marker, the method comprising:
i) providing DNA substrates that are operably linked to expression of the selectable marker, wherein each DNA substrate includes a segment comprising a DNA sequence configured to detect binding of a contiguous polypeptide comprising three distinct zinc fingers that are F1, F2, and F3, respectively, and wherein the F1, the F2 and the F3 are optionally in an N-terminal to C-terminal orientation;
ii) expressing the plurality of zinc fingers in a series of in vivo assays within cells that comprise the DNA substrates, wherein:
iii) expressing the plurality of zinc fingers in a series of in vivo assays within cells that comprise the DNA substrates, wherein:
a) in each assay the F1 comprises the same amino acid sequence;
b) in each assay the F2 comprises at least one amino acid difference relative to domain 1 of other zinc fingers in other assays in the series, and wherein said amino acid difference is optionally at position 6 of the F2, and wherein F1 and F2 comprise a functional pair in each assay; and
c) in each assay one or more of positions −1, 1, 2, 3, 5 and 6 of each F3 α-helix are randomized;
iv) selecting zinc fingers that promote expression of the selectable marker; and
v) determining the sequence of the zinc fingers that promote expression of the selectable marker to identify amino acids that promote said expression, wherein the identified amino acids that promote said expression are included in at least the F3 [domain 2].

2. The method of claim 1, wherein all of positions −1, 1, 2, 3, 5 and 6 of each F3 α-helix are randomized.

3. The method of claim 2, wherein said amino acid difference for each series of assays is at position 6 of the F2.

4. The method of claim 2, wherein each assay in the series of assays comprises at least 64 million distinct F3 α-helices.

5. The method claim 4, wherein a series of at least four assays are performed.

6. The method of claim 5, wherein the segment of the DNA substrate configured to detect the binding in each assay in the series comprises at least one variable segment, wherein the variable segment comprises three base pairs (bp) targets for use in determining the binding.

7. The method of claim 6, wherein 64 distinct 3 bp segments are included in each assay in the series.

8. The method of claim 7, wherein the series of assays is such that 64 independent selections can identify zinc fingers comprising a helices that may be able to interact with each of the 64 distinct 3 bp targets, and wherein sufficient selections are performed such that at least 16 billion unique zinc figure-DNA substrate interactions may be present and can be analyzed for expression of the selectable marker or the detectable marker or a combination thereof.

9. The method of claim 8, wherein the sufficient selections comprise 384 selections.

10. The method of claim 9, wherein the selectable marker is present and comprises HIS3.

11. The method of claim 9, wherein the detectable marker is present and comprises a fluorescent protein.

12. A zinc finger comprising an amino acid F3 [domain 2] sequence identified by claim 1.

13. A contiguous polypeptide comprising a set of zinc fingers, one of which comprises an amino acid sequence of an F3 identified by the method of claim 1, and a second of which comprises an F2 that was also present in an assay of the method of claim 1, and wherein said contiguous polypeptide can bind with specificity to a 3 bp segment of a DNA substrate that was also present in said assay.

14. A contiguous polypeptide comprising a set of zinc fingers, one of which comprises an amino acid sequence of an F3 identified by the method of claim 1, and a second of which comprises an F2 that was present in an assay of the method of claim 1, and wherein said contiguous polypeptide can bind with specificity to a 3 bp segment of a DNA substrate that was also present in said assay, and further comprising an F1 that was present in an assay of the method of claim 1, wherein said contiguous polypeptide can bind with specificity to the 3 bp segment of the DNA substrate.
